(19) United States
(12) Patent Application Publication
(10) Pub. No.: US 2021/0387968 A1
(43) Pub. Date: Dec. 16, 2021

(54) HETEROARYLMETHYLENE DERIVATIVES AS DNA POLYMERASE THETA INHIBITORS

(71) Applicant: IDEAYA Biosciences, Inc., South San Francisco, CA (US)

(72) Inventors: Hilary Plake BECK, South San Francisco, CA (US); Michael DILLON, South San Francisco, CA (US); Brian JONES, South San Francisco, CA (US); Luisruben P. MARTINEZ, South San Francisco, CA (US)

(21) Appl. No.: 17/426,436

(22) PCT Filed: Jan. 30, 2020
(86) PCT No.: PCT/US2020/015803

§ 371 Date: Jul. 28, 2021

Related U.S. Application Data
(60) Provisional application No. 62/799,500, filed on Jan. 31, 2019.

(51) Int. Cl. C07D 401/14; C07D 401/12; C07D 233/64

(52) U.S. Cl. CPC C07D 401/14; C07D 233/64; C07D 401/12

(57) ABSTRACT

Disclosed herein are certain acetamido derivatives that are DNA Polymerase Theta (Polθ) inhibitors of Formula (I). Also, disclosed are pharmaceutical compositions comprising such compounds and methods of treating diseases treatable by inhibition of Polθ such as cancer, including homologous recombination (HR) deficient cancers.

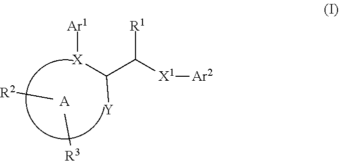

### CROSS-REFERENCES TO RELATED APPLICATIONS

This application claims the benefit under 35 U.S.C. 119(e) of U.S. Provisional Application No. 62/799,500, filed on Jan. 31, 2019, which is hereby incorporated herein by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

This application contains a Sequence Listing which has been submitted electronically in ASCII format and is hereby incorporated by reference in its entirety. Said ASCII copy, created on Jan. 27, 2020, is named 052326-519WO_SL_ST25.txt and is 998 bytes in size.

### BACKGROUND OF THE INVENTION

Targeting DNA repair deficiencies has become a proven and effective strategy in cancer treatment. However, DNA repair deficient cancers often become dependent on backup DNA repair pathways, which present an “Achilles heel” that can be targeted to eliminate cancer cells, and is the basis of synthetic lethality. Synthetic lethality is exemplified by the success of poly (ADP-ribose) polymerase (PARP) inhibitors in treating BRCA-deficient breast and ovarian cancers (Audeh M. W., et al., Lancet (2010); 376 (9737): 245-51).

DNA damage repair processes are critical for genome maintenance and stability, among which, double strand breaks (DSBs) are predominantly repaired by the nonhomologous end joining (NHEJ) pathway in G1 phase of the cell cycle and by homologous recombination (HR) in S-G2 phases. A less addressed alternative end-joining (alt-EJ), also known as microhomology-mediated end-joining (MMEJ) pathway, is commonly considered as a “backup” DSB repair pathway when NHEJ or HR are compromised. Numerous genetic studies have highlighted a role for polymerase theta (Polθ, encoded by POLQ) in stimulating MMEJ in higher organisms (Chan S. H., et al., PLoS Genet. (2010); 6: e1001005; Roerink S. F., et al., Genome research. (2014); 24: 954-962; Ceccaldi R., et. al., Nature (2015); 518: 258-62; and Mateos-Gomez P. A., et al., Nature (2015); 518: 254-57).

The identification of mammalian POLQ initially arose from interest in the POLQ ortholog Mus308 gene product of Drosophila melanogaster. Mus308 mutants are hypersensitive to agents that cause DNA inter-strand cross-links (ICL) (Aguirrezabalaga I., et al., Genetics. (1995); 139:649-658), which implied that Mus308 may play a specific role in repair of ICLs in DNA. Characterization of the POLQ gene showed that it encodes an unusual domain configuration, with a large central portion flanking by a N-terminal DNA helicase domain and a C-terminal DNA polymerase domain (Harris P. V., et al., Mol Cell Biol. (1996); 16: 5764-5771). The mechanisms by which Polθ polymerase functions in alt-EJ were also found to efficiently promote end-joining when overhangs contained >2 bp of microhomology were present (Kent T., et al., Elife (2016); 5: e13740), and Kent T., et al., Nat. Struct. Mol. Biol. (2015); 22: 230-237. On the other hand, the helicase domain of Polθ contributes to microhomology annealing (Chan S H et al., PLoS Genet. (2010); 6: e1001005; and Kawamura K et al., Int. J. Cancer (2004); 109: 9-16).

The expression of Polθ is largely absent in normal cells but upregulated in breast, lung, and ovarian cancers (Ceccaldi R., et al., Nature (2015); 518, 258-62). Additionally, the increase of Polθ expression correlates with poor prognosis in breast cancer (Lemee F et al., Proc Natl Acad Sci USA. (2010); 107: 13390-5). It has been shown that cancer cells with deficiency in HR, NHEJ or ATM are highly dependent on Polθ expression (Ceccaldi R., et al., Nature (2015); 518: 258-62, Mateos-Gomez P A et al., Nature (2015); 518: 254-57, and Wyatt D. W., et al., Mol. Cell (2016); 63: 662-73). Therefore, Polθ is an attractive target for novel synthetic lethal therapy in cancers containing DNA repair defects.

### BRIEF SUMMARY OF THE INVENTION

Disclosed herein are certain heteroarylmethylene derivatives that are DNA Polymerase Theta (Polθ) inhibitors, in particular inhibitors of polymerase domain of Polθ. Also, disclosed are pharmaceutical compositions comprising such compounds and methods of treating and/or preventing diseases treatable by inhibition of Polθ such as cancer, including homologous recombination (HR) deficient cancers.

In a first aspect, provided is a compound of Formula (I):

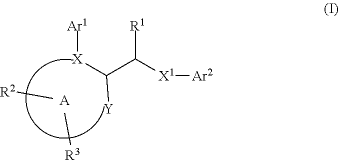

wherein:

X¹ is —NH— or —O—;

A is:

(i) a five membered heteroaryl ring wherein X is C or N, Y is O or N, and the heteroaryl may contain an additional nitrogen atom; or

(ii) a six-membered heteroaryl ring wherein X is C, Y is N, and the heteroaryl may contain one or two additional nitrogen atoms;

Ar¹ is phenyl, heteroaryl, or heterocyclyl, wherein each aforementioned ring is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂;

Ar² is phenyl or heteroaryl, wherein said phenyl and heteroaryl are substituted with R^{d} and further substituted with R^{e} and R^{f}, wherein R^{d} is haloalkyl, and R^{e} and R^{f} are independently selected from hydrogen, alkyl, halo, haloalkyl, alkoxy, haloalkoxy, hydroxy, cyano, cyanomethyl, aminocarbonylmethyl, heteroaryl, or heterocyclyl, wherein said heteroaryl and heterocyclyl of R^{e} and/or R^{f} are unsubstituted or substituted with one, two, or three substituents independently selected from alkyl, halo, haloalkyl, and hydroxy;

R¹ is hydrogen, alkyl, hydroxyalkyl, alkoxyalkyl, aminoalkyl, aminocarbonylalkyl, or phenalkyl, wherein phenyl in phenalkyl is substituted with R^{g}, R^{h}, and Rⁱ, wherein R^{g}, R^{h}, and Rⁱare independently selected from hydrogen, alkyl, halo, haloalkyl, alkoxy, haloalkoxy, hydroxy, and cyano; and

R² and R³ are independently hydrogen, alkyl, halo, haloalkyl, haloalkoxy, cyano, or —CONH₂; or

a pharmaceutically acceptable salt thereof, provided that the compound of Formula (I) is not:
2-pyridinamine, N-[(4-phenyl-4H-1,2,4-triazol-3-yl)methyl]-6-(trifluoromethyl)-; 5-(((5-bromo-6-(trifluoromethyl)pyridin-2-yl)oxy)methyl)-3-(chloromethyl)-4-(6-chloropyridin-3-yl)isoxazole;
2-pyrimidinamine, N-[(4-phenyl-4H-1,2,4-triazol-3-yl)methyl]-4-(trifluoromethyl)-;
2-pyridinamine, N-[(4-phenyl-4H-1,2,4-triazol-3-yl)methyl]-5-(trifluoromethyl)-;
pyrimidine, 4-[[[3-fluoro-5-(trifluoromethyl)-2-pyridinyl]oxy]methyl]-5-phenyl-;
1,4′-bi-1H-pyrazole, 4-iodo-1′-methyl-5′-[[4-(trifluoromethyl)phenoxy]methyl]-;
3H-1,2,4-triazol-3-one, 2,4-dihydro-5-methoxy-2-methyl-4-[2-[1-[[4-(trifluoromethyl)-2-pyridinyl]oxy]ethyl]-3-pyridinyl]-;
3(2H)-isoxazolone, 5-methoxy-2-methyl-4-[2-[1-[3-(trifluoromethyl)phenoxy]ethyl]-3-pyridinyl]-;
3H-1,2,4-triazol-3-one, 2,4-dihydro-5-methoxy-2-methyl-4-[2-[[[4-(trifluoromethyl)-2-pyridinyl]oxy]methyl]-3-pyridinyl]-;
3(2H)-isoxazolone, 5-methoxy-2-methyl-4-[2-[[3-(trifluoromethyl)phenoxy]methyl]-3-pyridinyl]-;
3(2H)-isoxazolone, 5-methoxy-2-methyl-4-[2-[1-[[4-(trifluoromethyl)-2-pyridinyl]oxy]ethyl]-3-pyridinyl]-;
3(2H)-isoxazolone, 5-methoxy-2-methyl-4-[2-[[[4-(trifluoromethyl)-2-pyridinyl]oxy]methyl]-3-pyridinyl]-;
3H-1,2,4-triazol-3-one, 2,4-dihydro-5-methoxy-2-methyl-4-[2-[1-[3-(trifluoromethyl)phenoxy]ethyl]-3-pyridinyl]-;
3H-1,2,4-triazol-3-one, 2,4-dihydro-5-methoxy-2-methyl-4-[2-[[3-(trifluoromethyl)phenoxy]methyl]-3-pyridinyl]-; or
4-pyrimidinamine, N-[(5-phenyl-4-oxazolyl)methyl]-2-(2-pyridinyl)-6-(trifluoromethyl)-;
   or a pharmaceutically acceptable salt thereof

In a second aspect, provided is a pharmaceutical composition comprising a compound of Formula (I) or a pharmaceutically acceptable thereof and at least one pharmaceutically acceptable excipient.

In a third aspect, provided is a method for treating and/or preventing a disease characterized by overexpression of Polθ in a patient comprising administering to the patient a therapeutically effective amount of:

(a) a compound of Formula (II):

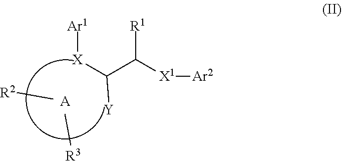

wherein:

X¹ is —NH— or —O—;

A is:

(i) a five membered heteroaryl ring wherein X is C or N, Y is O or N, and the heteroaryl may contain an additional nitrogen atom; or

(ii) a six-membered heteroaryl ring wherein X is C, Y is N, and the heteroaryl may contain one or two additional nitrogen atoms;

Ar¹ is phenyl, phenylalkyl, heteroaryl, heteroaralkyl, heterocyclyl, or heterocyclylalkyl wherein each aforementioned rings is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂;

Ar² is phenyl, heteroaryl, or fused heteroaryl wherein each aforementioned ring is substituted with R, R^{d}, R^{e} and R^{f}, wherein R is hydrogen or halo, and R^{d}, R^{e} and R^{f} are independently selected from hydrogen, alkyl, halo, haloalkyl, alkoxy, haloalkoxy, hydroxy, cyano, cyanomethyl, aminocarbonylmethyl, heteroaryl, or heterocyclyl wherein said heteroaryl and heterocyclyl of R^{d}, R^{e} and R^{f} are unsubstituted or substituted with one, two, or three substituents independently selected from alkyl, halo, haloalkyl, and hydroxy;

R¹ is hydrogen, alkyl, hydroxyalkyl, alkoxyalkyl, aminoalkyl, aminocarbonylalkyl, or phenalkyl, wherein phenyl in phenalkyl is substituted with R^{g}, R^{h}, and/or Rⁱ, wherein R^{g}, R^{h}, and Rⁱ are independently selected from alkyl, halo, haloalkyl, alkoxy, haloalkoxy, hydroxy, and cyano; and

R² and R³ are independently hydrogen, alkyl, alkoxy, hydroxy, halo, haloalkyl, haloalkoxy, cyano, or —CONH₂; (preferably R² and R³ are independently hydrogen, alkyl, halo, haloalkyl, haloalkoxy, cyano, or —CONH₂) or

(b) a compound of Formula (I); or

a pharmaceutically acceptable salt thereof (or an embodiment thereof disclosed herein).

In first embodiment of the third aspect, the patient is in recognized need of such treatment. In second embodiment of the third aspect and first embodiment contained therein, the compound of Formula (I) or (II) (or an embodiment thereof disclosed herein), or a pharmaceutically acceptable salt thereof is administered in a pharmaceutical composition. In third embodiment of the third aspect and first and second embodiments contained therein, the disease is a cancer.

In a fourth aspect, provided is a method of treating and/or preventing a homologous recombinant (HR) deficient cancer in a patient comprising administering to the patient a therapeutically effective amount of a compound of Formula (I) or (II) (or an embodiment thereof disclosed herein), or a pharmaceutically acceptable salt thereof. In first embodiment of the fourth aspect, the patient is in recognized need of such treatment. In second embodiment of the fourth aspect and first embodiment contained therein, the compound of Formula (I) or (II) (or an embodiment thereof disclosed herein), or a pharmaceutically acceptable salt thereof is administered in a pharmaceutical composition.

In a fifth aspect, provided is a method for inhibiting DNA repair by Polθ in a cancer cell comprising contacting the cell with an effective amount of a compound of Formula (I) or (II) (or an embodiment thereof disclosed herein), or a pharmaceutically acceptable salt thereof. In a first embodiment, the cancer is HR deficient cancer.

In a sixth aspect, provided is a method for treating and/or preventing a cancer in a patient, wherein the cancer is characterized by a reduction or absence of BRCA gene expression, the absence of the BRAC gene, or reduced function of BRCA protein, comprising administering to the subject a therapeutically effective amount of a compound of Formula (I) or (II) (or an embodiment thereof disclosed herein), or a pharmaceutically acceptable salt thereof optionally in a pharmaceutical composition.

In a seventh aspect, provided is a compound of Formula (I) or (II) (or an embodiment thereof disclosed herein), or a pharmaceutically acceptable salt thereof for inhibiting DNA repair by Polθ in a cell. In a first embodiment, the cell is HR deficient cell.

In an eighth aspect, provided is a compound of Formula (I) or (II) (or an embodiment thereof disclosed herein), or a pharmaceutically acceptable salt thereof for use in the treatment and/or prevention of a disease in a patient, wherein the disease is characterized by overexpression of Polθ.

In a ninth aspect, provided is a compound of Formula (I) or (II) (or an embodiment thereof disclosed herein), or a pharmaceutically acceptable salt thereof for use in the treatment and/or prevention of a cancer in a patient, wherein the cancer is characterized by a reduction or absence of BRAC gene expression, the absence of the BRAC gene, or reduced function of BRAC protein.

In a tenth aspect, provided is a compound of Formula (I) or (II) (or an embodiment thereof disclosed herein), or a pharmaceutically acceptable salt thereof for use in the treatment and/or prevention of a HR deficient cancer in a patient.

In an eleventh aspect, provided is a compound of Formula (I) or (II) (or any embodiment thereof disclosed herein), or a pharmaceutically acceptable salt thereof for use in the treatment or prevention of a cancer that is resistant to poly(ADP-ribose)polymerase (PARP) inhibitor therapy in a patient. Examples of cancers that are resistant to PARP-inhibitors include, but are not limited to, breast cancer, ovarian cancer, lung cancer, bladder cancer, liver cancer, head and neck cancer, pancreatic cancer, gastrointestinal cancer and colorectal cancer.

In any of the third to eleventh aspect, the cancer is lymphoma, soft tissue, rhabdoid, multiple myeloma, uterus, gastric, peripheral nervous system, rhabdomyosarcoma, bone, colorectal, mesothelioma, breast, ovarian, lung, fibroblast, central nervous system, urinary tract, upper aerodigestive, leukemia, kidney, skin, esophagus, and pancreas (data from large scale drop out screens in cancer cell lines indicate that some cell lines from the above cancers are dependent on polymerase theta for proliferation see https://depmap.org/portal/).

In first embodiment, a HR-deficient cancer is breast cancer. Breast cancer includes, but is not limited to, lobular carcinoma in situ, a ductal carcinoma in situ, an invasive ductal carcinoma, triple negative, HER positive, estrogen receptor positive, progesterone receptor positive, HER and estrogen receptor positive, HER and estrogen and progesterone receptor, positive inflammatory breast cancer, Paget disease of nipple, Phyllodes tumor, angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, and mixed carcinoma. In second embodiment, HR-deficient cancer is ovarian cancer. Ovarian can includes, but is not limited to, epithelial ovarian carcinomas, maturing teratomas, dysgerminomas, endodermal sinus tumors, granulosa-theca tumors, Sertoli-Leydig cell tumors, and primary peritoneal carcinoma.

In a twelfth aspect, provided herein is a method of identifying Polθ polymerase domain inhibitory activity in a test compound, said method comprising
(i) contacting the test compound and Polθ polymerase domain (residues 1819-2590) in an assay buffer to form a reaction pre-mixture;
(ii) contacting the reaction pre-mixture of (i) with (a) a dNTP substrate mixture, and
   (b) a primed molecular beacon DNA to form a test solution,
   wherein the primed molecular beacon DNA comprises a labeled template annealed to a primer, wherein the labeled template is SEQ ID NO: 1 (5′-CCTTCCTCCCGTGTCTTGTACCTTCCCGTCAGGAGGAAGG-3′) having one or more fluorescent labels, and the primer is SEQ ID NO: 3 (5′-GACGGGAAGG-3′); and
(iii) measuring fluorescence intensity of the test reaction mixture, wherein said method further comprises performing steps (i)-(iii) with a positive control sample represented by Formula (I) or (II) (or any embodiments thereof).

In some embodiments, the final concentration of Polθ polymerase domain in the test reaction mixture is 4 nM.

In some embodiments, the assay buffer is 20 m M TRIS, pH 7.80, 50 mM KCl, 10 mM MgCl₂, 1 mM DTT, 0.01% BSA, 0.01% Tween20.

In some embodiments, the dNTP substrate mixture is an equal mixture of each natural dNTP (dTTP, dATP, dCTP, and dGTP). In some embodiments the dNTP in the substrate mixture is 48 μM.

In some embodiments the labeled template is fluorescently labeled with one or more fluorescent labels. A number of fluorescent labels (and quenchers) are known in the art. In some embodiments the one or more fluorescent labels comprise 5′-TAMRA and 3′-BHQ. In some embodiments the sequence of the labeled template is SEQ ID NO 2: 5′-CCTTCCTCCCGTGTCTTGTACCTTCCCGTCAGGAGGAAGG-3′ with 5′-TAMRA and 3′-BHQ.

In some embodiments the primed molecular beacon DNA further comprises a priming buffer. In some embodiments, the buffer is 10 mM Tris-HCl pH 8.0, 100 mM NaCl buffer, and the concentration of the primed molecular beacon DNA is 96 nM.

A person of skill in the art will recognize that the fluorescence measured will depent on the labels being used in the assay. In some embodiments, absorbance (λₑₓ=485 nm, λₑₘ=535 nm) of the Pol theta reaction mixture.

### BRIEF DESCRIPTION OF THE DRAWINGS

NOT APPLICABLE

### DETAILED DESCRIPTION OF THE INVENTION

Before the present invention is further described, it is to be understood that the invention is not limited to the particular embodiments set forth herein, and it is also to be understood that the terminology used herein is for the purpose of describing particular embodiments only, and is not intended to be limiting.

The singular forms “a,” “an,” and “the” as used herein and in the appended claims include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

When needed, any definition herein may be used in combination with any other definition to describe a composite structural group. By convention, the trailing element of any such definition is that which attaches to the parent moiety. For example, the composite group alkoxyalkyl means that an alkoxy group is attached to the parent molecule through an alkyl group.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with one alkoxy group, as defined above, e.g., 2-methoxyethyl, 1-, 2-, or 3-methoxypropyl, 2-ethoxyethyl, and the like.

“Alkylcarbonyl” means a —C(O)R radical where R is alkyl as defined herein, e.g., methylcarbonyl, ethylcarbonyl, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, haloalkyl, hydroxyalkyl, alkoxyalkyl, or alkylcarbonyl, each as defined herein, e.g., aminomethyl, aminoethyl, methylaminomethyl, and the like.

“Aminocarbonylalkyl” means a -(alkylene)-CONH₂ radical wherein alkylene as defined herein, e.g., aminocarbonylmethyl, aminocarbonylethyl, aminocarbonylethyl, and the like. When the group is —CH₂CONH₂, it may be referred to herein as aminocarbonylmethyl.

“Aryl” means a monovalent monocyclic or bicyclic aromatic hydrocarbon radical of 6 to 10 ring atoms e.g., phenyl or naphthyl.

“Phenalkyl” of “phenylalkyl” means a -(alkylene)-R radical where R is phenyl e.g., benzyl, phenethyl, and the like.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Cycloalkyloxy or Cycloalkoxy” means —O—R radical where R is cycloalkyl as defined above. Examples include, but are not limited to, cyclopropyloxy, cyclobutyloxy, and the like.

“Fused heteroaryl” means a six-membered heteroaryl ring fused to a three to six membered saturated cycloalkyl, each ring as defined herein.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon, unless stated otherwise. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaralkyl” means a -(alkylene)-R radical where R is heteroaryl, each group as defined herein.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic.

“Heterocyclylalkyl” means a -(alkylene)-R radical where R is heterocyclyl, each group as defined herein.

“Oxo,” as used herein, alone or in combination, refers to ═(O).

“Pharmaceutically acceptable salts” as used herein is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The present disclosure also includes protected derivatives of compounds of the present disclosure. For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes prodrugs of the compound of Formula (I) or (II) (and any embodiment thereof disclosed herein including specific compounds) or a pharmaceutically acceptable salt thereof. Prodrugs of the compounds described herein are those compounds that readily undergo chemical changes under physiological conditions to provide the compounds of the present invention. An example, without limitation, of a prodrug would be a compound which is administered as an ester (the “prodrug”), but then is metabolically hydrolyzed to the carboxylic acid, the active entity. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of Formulae (I) and (II) (and any embodiment thereof disclosed herein including specific compounds) can exist in unsolvated forms as well as solvated forms, including hydrated forms. In general, the solvated forms are equivalent to unsolvated forms and are intended to be encompassed within the scope of the present invention. Certain compounds of Formulae (I) and (II) may exist in multiple crystalline or amorphous forms. In general, all physical forms are equivalent for the uses contemplated by the present disclosure and are intended to be within the scope of the present disclosure.

Certain compounds of Formulae (I) and (II) (and any embodiment thereof disclosed herein including specific compounds) possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of Formulae (I) and (II) (and any embodiment thereof disclosed herein including specific compounds) may also contain unnatural amounts of isotopes at one or more of the atoms that constitute such compounds. Unnatural amounts of an isotope may be defined as ranging from the amount found in nature to an amount 100% of the atom in question. Exemplary isotopes that can be incorporated into compounds of the present invention, such as a compound of Formula (I) and (II) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” and as used herein includes all mammals including humans. Examples of patients include humans, livestock such as cows, goats, sheep, pigs, and rabbits, and companion animals such as dogs, cats, rabbits, and horses. Preferably, the patient is a human.

“In need of treatment” as used herein means the patient is being treated by a physician or other caregiver after diagnoses of the disease. For example, the patient has been diagonosed as having a disease linked to overexpression of Polθ or a homologous recombination (HR)-deficient cancer.

“Administration”, “administer” and the like, as they apply to, for example, a patient, cell, tissue, organ, or biological fluid, refer to contact of, for example, a compound of Formula (I), a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Therapeutically effective amount” as used herein means the amount of a compound of Formula (I) or (II) (and any embodiment thereof disclosed herein including specific compounds) or a pharmaceutically acceptable salt thereof that, when administered to a patient for treating a disease either alone or as part of a pharmaceutical composition and either in a single dose or as part of a series of doses, is sufficient to affect such treatment for the disease. The “therapeutically effective amount” will vary depending on the compound, the disease and its severity and the age, weight, etc., of the mammal to be treated. The therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I) (or, e.g., a metabolite thereof) at a particular time post-administration may be indicative of whether a therapeutically effective amount has been used.

“Treating” or “treatment” of a disease includes:

(1) inhibiting the disease, i.e., arresting or reducing the development of the disease or its clinical symptoms; or

(2) relieving the disease, i.e., causing regression of the disease or its clinical symptoms.

“Inhibiting”, “reducing,” or any variation of these terms in relation of Polθ, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of Polθ activity compared to its normal activity.

The term “preventing” refers to causing the clinical symptoms of the disease not to develop in a mammal that may be exposed to or predisposed to the disease but does not yet experience or display symptoms of the disease.

The term “homologous recombination” refers to the cellular process of genetic recombination in which nucleotide sequences are exchanged between two similar or identical DNA.

The term “homologous recombination (HR) deficient cancer” refers to a cancer that is characterized by a reduction or absence of a functional HR repair pathway. HR deficiency may arise from absence of one or more HR-associated genes or presence of one or more mutations in one or more HR-associated genes. Examples of HR-associated genes include BRCA1, BRCA2, RAD54, RAD51B, Ct1P (Choline Transporter-Like Protein), PALB2 (Partner and Localizer of BRCA2), XRCC2 (X-ray repair complementing defective repair in Chinese hamster cells 2), RECQL4 (RecQ Protein-Like 4), BLM (Bloom syndrome, RecQ helicase-like), WRN (Werner syndrome, one or more HR-associated genes) Nbs 1 (Nibrin), and genes encoding Fanconi anemia (FA) proteins or FA-like genes e.g, FANCA, FANCB, FANCC, FANCD1 (BRCA2), FANCD2, FANCE, FANCF, FANCG, FANCI, FANJ (BRIPI), FANCL, FANCM, FANCN (RALB2), FANCP (SLX4), FANCS (BRCA1), RAD51C, and XPF.

The term “Polθ overexpression” refers to the increased expression or activity of Polθ in a diseased cell e.g., cancerous cell, relative to expression or activity of Polθ in a control cell (e.g., non-diseased cell of the same type). The amount of Polθ overexpression can be at least 2-fold, at least 3-fold, at least 4-fold, at least 5-fold, at least 10-fold, at least 20-fold, at least 50-fold, at least 100-fold, at least 500-fold, or at least 1000-fold relative to Polθ expression in a control cell. Examples of Polθ overexpressing cancers include, but are not limited to, certain ovarian, breast, cervical, lung, colorectal, gastric, bladder, and prostate cancers.

Representative compound of Formula (I) and (II) are listed in Table 1 below:

| Cpd. | | |
|---|---|---|
| # | Structure | Name |
| 1 | 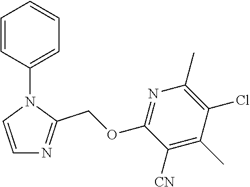 [SMILES: Cc1nc(OCc2nccn2-c2ccccc2)c(C#N)c(C)c1Cl] | 5-chloro-4,6-dimethyl-2-((1-phenyl- 1H-imidazol-2-yl)methoxy)- nicotinonitrile |
| 2 | 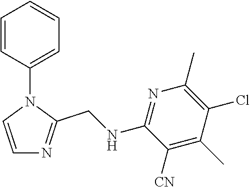 [SMILES: Cc1nc(NCc2nccn2-c2ccccc2)c(C#N)c(C)c1Cl] | 5-chloro-4,6-dimethyl-2-((1-phenyl- 1H-imidazol-2-yl)methylamino)- nicotinonitrile |
| 3 | 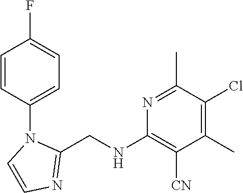 [SMILES: Cc1nc(NCc2nccn2-c2ccc(F)cc2)c(C#N)c(C)c1Cl] | 5-chloro-2-(((1-(4-fluorophenyl)-1H- imidazol-2-yl)methyl)amino)-4,6- dimethyl-nicotinonitrile |
| 4 | 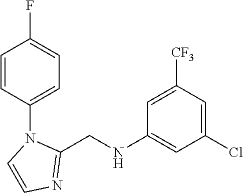 [SMILES: Fc1ccc(-n2ccnc2CNc2cc(Cl)cc(C(F)(F)F)c2)cc1] | 3-chloro-N-((1-(4-fluorophenyl)-1H- imidazol-2-yl)methyl)-5- (trifluoromethyl)-benzenamine |
| 5 | 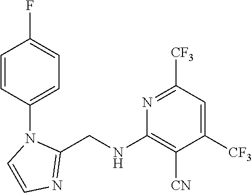 [SMILES: N#Cc1c(C(F)(F)F)cc(C(F)(F)F)nc1NCc1nccn1-c1ccc(F)cc1] | 2-([[1-(4-fluorophenyl)-1H-imidazol-2- yl]methyl]amino)-4,6- bis(trifluoromethyl)-pyridine-3- carbonitrile |
| 6 | 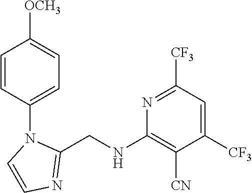 [SMILES: COc1ccc(-n2ccnc2CNc2nc(C(F)(F)F)cc(C(F)(F)F)c2C#N)cc1] | 2-([[1-(4-methoxyphenyl)-1H- imidazol-2-yl]methyl]amino)-4,6- bis(trifluoromethyl)-pyridine-3- carbonitrile |
| 7 | 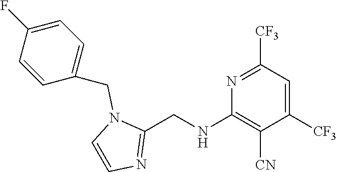 [SMILES: N#Cc1c(C(F)(F)F)cc(C(F)(F)F)nc1NCc1nccn1Cc1ccc(F)cc1] | 2-[([1-[(4-fluorophenyl)methyl]-1H- imidazol-2-yl]methyl)amino]-4,6-bis- (trifluoromethyl)pyridine-3-carbonitrile |
| 8 | 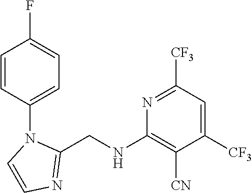 [SMILES: N#Cc1c(C(F)(F)F)cc(C(F)(F)F)nc1NCc1nccn1-c1ccc(F)cc1] | 2-[[1-(4-fluorophenyl)-1H-imidazol-2- yl]methoxy]-4,6-bis(trifluoromethyl)- pyridine-3-carbonitrile |
| 9 | 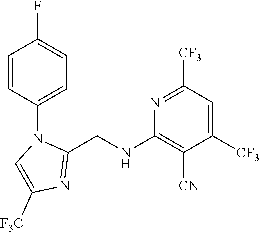 [SMILES: N#Cc1c(C(F)(F)F)cc(C(F)(F)F)nc1NCc1nc(C(F)(F)F)cn1-c1ccc(F)cc1] | 2-([[1-(4-fluorophenyl)-4- (trifluoromethyl)imidazol-2- yl]methyl]amino)-4,6- bis(trifluoromethyl)benzonitrile |
| 10 | 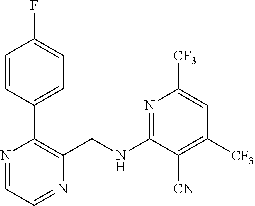 [SMILES: N#Cc1c(C(F)(F)F)cc(C(F)(F)F)nc1NCc1nccnc1-c1ccc(F)cc1] | 2-((3-(4-fluorophenyl)pyrazin-2- yl)methylamino)-4,6- bis(trifluoromethyl)-nicotinonitrile |
| 11 | 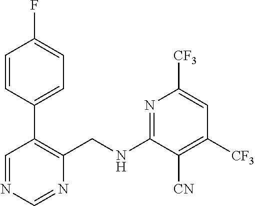 [SMILES: N#Cc1c(C(F)(F)F)cc(C(F)(F)F)nc1NCc1ncncc1-c1ccc(F)cc1] | 2-((5-(4-fluorophenyl)pyrimidin-4- yl)methylamino)-4,6- bis(trifluoromethyl)-nicotinonitrile |
| 12 | 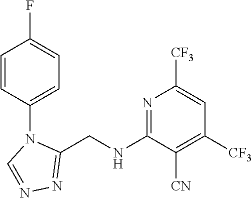 [SMILES: N#Cc1c(C(F)(F)F)cc(C(F)(F)F)nc1NCc1nncn1-c1ccc(F)cc1] | 2-((4-(4-fluorophenyl)-4H-1,2,4- triazol-3-yl)methylamino)-4,6- bis(trifluoromethyl)-nicotinonitrile |
| 13 | 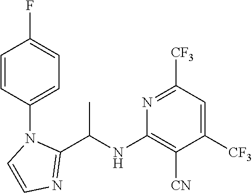 [SMILES: CC(Nc1nc(C(F)(F)F)cc(C(F)(F)F)c1C#N)c1nccn1-c1ccc(F)cc1] | 2-([1-[1-(4-fluorophenyl)imidazol-2- yl]ethyl]amino)-4,6- bis(trifluoromethyl)pyridine-3- carbonitrile |
| 14 | 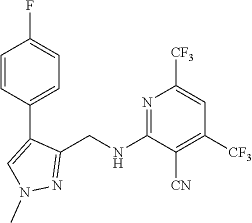 [SMILES: Cn1cc(-c2ccc(F)cc2)c(CNc2nc(C(F)(F)F)cc(C(F)(F)F)c2C#N)n1] | 2-(((4-(4-fluorophenyl)-1-methyl-1H- pyrazol-3-yl)methyl)amino)-4,6- bis(trifluoromethyl)nicotinonitrile |
| 15 | 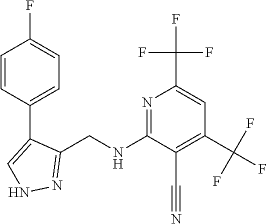 [SMILES: N#Cc1c(C(F)(F)F)cc(C(F)(F)F)nc1NCc1n[nH]cc1-c1ccc(F)cc1] | 2-(((4-(4-fluorophenyl)-1H-pyrazol-3- yl)methyl)amino)-4,6- bis(trifluoromethyl)-nicotinonitrile |
| 16 | 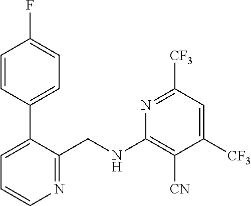 [SMILES: N#Cc1c(C(F)(F)F)cc(C(F)(F)F)nc1NCc1ncccc1-c1ccc(F)cc1] | 2-(((3-(4-fluorophenyl)pyridin-2- yl)methyl)amino)-4,6- bis(trifluoromethyl)-nicotinonitrile |
| 17 | 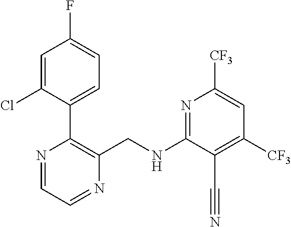 [SMILES: N#Cc1c(C(F)(F)F)cc(C(F)(F)F)nc1NCc1nccnc1-c1ccc(F)cc1Cl] | 2-([[3-(2-chloro-4-fluorophenyl)- pyrazin-2-yl]methyl]amino)-4,6- bis(trifluoromethyl)pyridine-3- carbonitrile |
| 18 | 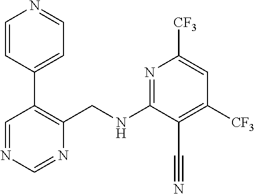 [SMILES: N#Cc1c(C(F)(F)F)cc(C(F)(F)F)nc1NCc1ncncc1-c1ccncc1] | 2-([[5-(pyridin-4-yl)pyrimidin-4- yl]methyl]amino)-4,6-bis(trifluoro- methyl)pyridine-3-carbonitrile |
| 19 | 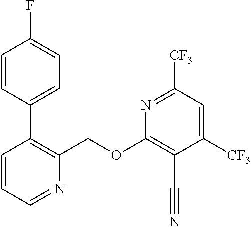 [SMILES: N#Cc1c(C(F)(F)F)cc(C(F)(F)F)nc1OCc1ncccc1-c1ccc(F)cc1] | 2-[[3-(4-fluorophenyl)pyridin-2- yl]methoxy]-4,6- bis(trifluoromethyl)pyridine-3- carbonitrile |
| 20 | 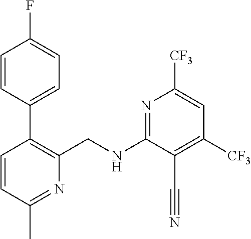 [SMILES: Cc1ccc(-c2ccc(F)cc2)c(CNc2nc(C(F)(F)F)cc(C(F)(F)F)c2C#N)n1] | 2-([[3-(4-fluorophenyl)-6- methylpyridin-2-yl]methyl]amino)-4,6- bis(trifluoromethyl)pyridine-3- carbonitrile |
| 21 | 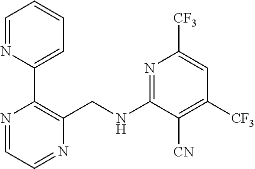 [SMILES: N#Cc1c(C(F)(F)F)cc(C(F)(F)F)nc1NCc1nccnc1-c1ccccn1] | 2-(((3-(pyridin-2-yl)pyrazin-2- yl)methyl)amino)-4,6- bis(trifluoromethyl)-nicotinonitrile |
| 22 | 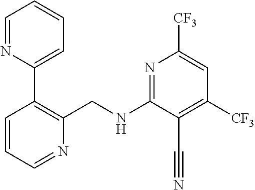 [SMILES: N#Cc1c(C(F)(F)F)cc(C(F)(F)F)nc1NCc1ncccc1-c1ccccn1] | 2-[[2,3′-bipyridine]-2′-carboximidoyl]- 4,6-bis(trifluoromethyl)-1,2- dihydropyridine-3-carbonitrile |
| 23 | 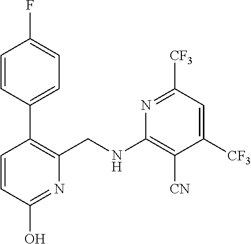 [SMILES: N#Cc1c(C(F)(F)F)cc(C(F)(F)F)nc1NCc1nc(O)ccc1-c1ccc(F)cc1] | 2-([[3-(4-fluorophenyl)-6-oxo-1H- pyridin-2-yl]methyl]amino)-4,6- bis(trifluoro-methyl)pyridine-3- carbonitrile |
| 24 | 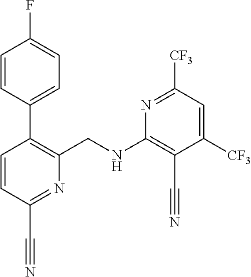 [SMILES: N#Cc1ccc(-c2ccc(F)cc2)c(CNc2nc(C(F)(F)F)cc(C(F)(F)F)c2C#N)n1] | 6-([[3-cyano-4,6- bis(trifluoromethyl)pyridin-2- yl]amino]methyl)-5-(4- fluorophenyl)pyridine-2- carbonitrile |
| 25 | 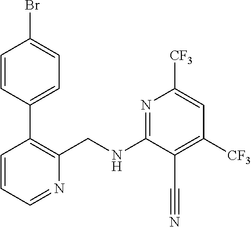 [SMILES: N#Cc1c(C(F)(F)F)cc(C(F)(F)F)nc1NCc1ncccc1-c1ccc(Br)cc1] | 2-([[3-(4-bromophenyl)pyridin-2- yl]methyl]amino)-4,6- bis(trifluoromethyl)-pyridine-3- carbonitrile |
| 26 | 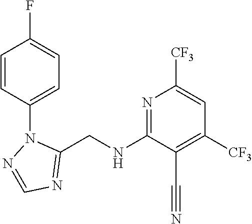 [SMILES: N#Cc1c(C(F)(F)F)cc(C(F)(F)F)nc1NCc1ncnn1-c1ccc(F)cc1] | 2-(((1-(4-fluorophenyl)-1H-1,2,4- triazol-5-yl)methyl)amino)-4,6- bis(trifluoro-methyl)nicotinonitrile |
| 27 | 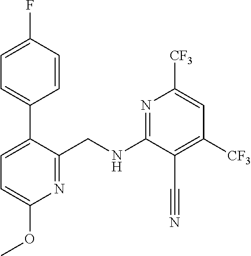 [SMILES: COc1ccc(-c2ccc(F)cc2)c(CNc2nc(C(F)(F)F)cc(C(F)(F)F)c2C#N)n1] | 2-([[3-(4-fluorophenyl)-6- methoxypyridin-2-yl]methyl]amino)- 4,6-bis(trifluoromethyl)pyridine-3- carbonitrile |

### Embodiments

In further embodiments 1 to 10 below, the present disclosure includes:

1. In embodiment 1, provided is a compound of Formula (I), or a pharmaceutically acceptable salt thereof, where R¹, R², R³, X¹, ring A, Ar¹, and Ar² are as described in the Summary above.

2. In embodiment 2, provided is a compound of Formula (II), or a pharmaceutically acceptable salt thereof, where R¹, R², R³, X¹, ring A, Ar¹, and Ar² are as described in the Summary above.

3. In embodiment 3, the compound of embodiment 1 or 2, or a pharmaceutically acceptable salt thereof, is wherein Ar² is a six- to ten-membered heteroaryl substituted with R^{d} and R^{e} and R^{f}, where R^{d} is haloalkyl.

4. In embodiment 4, the compound of embodiment 1 or 2, or a pharmaceutically acceptable salt thereof, is wherein Ar² is a six-membered heteroaryl substituted with R^{d} and R and R^{f}, where R^{d} is haloalkyl. In a first subembodiment of embodiment 4, Ar² is pyridinyl substituted with R^{d} and R^{e} and R^{f}, where R^{d} is haloalkyl. In a second subembodiment of embodiment 4, Ar² is pyridinyl substituted with R^{d} and R^{e} and R^{f}, where R^{d} is difluoromethyl or trifluoromethyl. In a third subembodiment of embodiment 4, Ar² is pyridin-2-yl substituted with R^{d} and R^{e} and R^{f}, where R^{d} is difluoromethyl or trifluoromethyl, R^{e} is haloalkyl, alkoxy, halo, haloalkoxy, hydroxy, or cyano, and R^{f} is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{f} are unsubstituted or substituted with one, two, or three substituents independently selected from alkyl, halo, haloalkyl, and hydroxy. In a fourth subembodiment of embodiment 4, Ar² is 4,6-di-trifluoromethylpyridin-2-yl, 3-cyano-4,6-di-trifluoromethylpyridin-2-yl, or 4,6-di-trifluoromethylpyrimidin-2-yl.

5. In embodiment 5, the compound of embodiment 1 or 2, or a pharmaceutically acceptable salt thereof, is wherein Ar² is phenyl substituted with R^{d} and R^{e} and R^{f}, where R^{d} is haloalkyl. In a first subembodiment of embodiment 5, Ar² is phenyl substituted with R^{d} and R^{e} and R^{f}, where R^{d} is difluoromethyl or trifluoromethyl. In a second subembodiment of embodiment 5, Ar² is phenyl substituted with R^{d} and R^{e} and/or R^{f}, where R^{d} is difluoromethyl or trifluoromethyl, R^{e} is haloalkyl, alkoxy, halo, haloalkoxy, hydroxy, or cyano, and R^{f} is hydrogen, alkyl, halo, haloalkyl, alkoxy, haloalkoxy, hydroxy, cyano, cyanomethyl, aminocarbonylmethyl, heteroaryl, and heterocyclyl wherein said heteroaryl and heterocyclyl of R^{f} are unsubstituted or substituted with one, two, or three substituents independently selected from alkyl, halo, haloalkyl, and hydroxy. In a fourth subembodiment of embodiment 5, Ar² is 3-chloro-5-trifluoromethylphenyl, 3-chloro-6-cyano-5-trifluoromethylphenyl, or 3,5-ditrifluoromethylphenyl.

6. In embodiment 6, the compound of embodiment 2, or a pharmaceutically acceptable salt thereof, is wherein Ar² is phenyl, fused heteroaryl, or six- to ten-membered heteroaryl, wherein each of the aforementioned rings are substituted with R, R^{d}, R^{e}, and R^{f}. In a first subembodiment of embodiment 6, Ar² is phenyl substituted with R, R^{d}, R^{e} and R^{f}. In a second subembodiment of embodiment 6, Ar² is six- to ten-membered heteroaryl substituted with R, R^{d}, R^{e}, and R^{f}. In a third subembodiment of embodiment 6, Ar² is 4-chloro-2-cyano-3,6-dimethylphenyl, 4-cyano-1-methylisoquinolin-3-yl, 3-bromo-5-chlorophenyl, 5-chloro-3-cyano-4,6-dimethylpyridin-2-yl, 3,5-dichloro-4,6-dimethylpyridin-2-yl, 4-cyano-6,7-dihydro-5H-cyclopenta[c]pyridine-2-yl, 3-cyano-4-methyl-6,7-dihydro-5H-cyclopenta[b]pyridin-2-yl, 4-cyano-1-methyl-6,7-dihydro-5H-cyclopenta-[c]pyridin-2-yl, 3-cyano-4-methylquinolin-2-yl, 3,5-dichlorophenyl, 5-chloro-4,6-dimethylpyridin-2-yl, 3-cyano-5-chloro-4-methylpyridin-2-yl, 3-cyano-5-chloro-6-methylpyridin-2-yl, or 3-cyano-5-chloro-4,6-dimethylpyridin-2-yl.

7. In embodiment 7, the compound of any one of embodiments 1 to 6 (and embodiments and subembodiments contained therein), or a pharmaceutically acceptable salt thereof, is wherein R¹ is hydrogen, methyl, hydroxymethyl, 2-hydroxyethyl, 4-hydroxybenzyl, or aminocarbonylmethyl. In a first subembodiment of embodiment 7, R¹ is hydrogen.

8. In embodiment 8, the compound of any one of embodiments 1 to 7 (and embodiments and subembodiments contained therein), or a pharmaceutically acceptable salt thereof, is wherein ring A is a five-membered heteroaryl ring. In a first subembodiment of embodiment 8, ring A is a ring of formula (i) to (v):

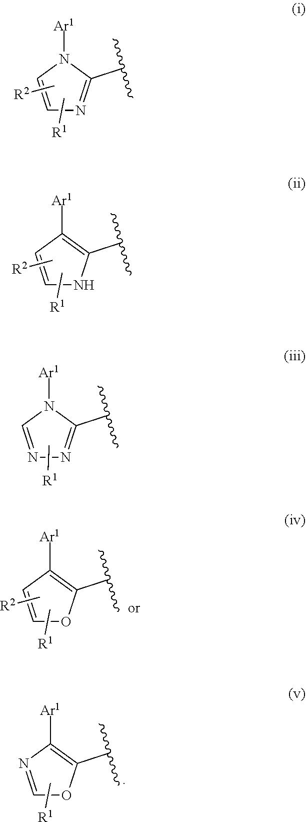

In first embodiment of first subembodiment, ring A has formula (i). In second embodiment of first subembodiment, ring A has formula (ii). In third embodiment of first subembodiment, ring A has formula (iii). In fourth embodiment of first subembodiment, ring A has formula (iv). In fifth embodiment of first subembodiment, ring A has formula (v).

9. In embodiment 9, the compound of any one of embodiments 1 to 7 (and embodiments and subembodiments therein), or a pharmaceutically acceptable salt thereof, is wherein ring A is a six-membered heteroaryl ring. In a first subembodiment of embodiment 9, ring A is a ring of formula (ia) to (ic):

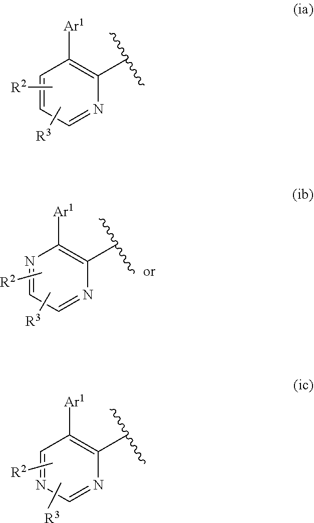

In first embodiment of first subembodiment, ring A has formula (ia). In second embodiment of first subembodiment, ring A has formula (ib). In third embodiment of first subembodiment, ring A has formula (ic).

10. In embodiment 10, the compound of any one of embodiments 1 to 9 (and embodiments and subembodiments contained therein), or a pharmaceutically acceptable salt thereof, is wherein Ar¹ is phenyl, wherein said phenyl is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂. In a first subembodiment of embodiment 10, Ar¹ is substituted with R^{a}, R^{b}, and R^{c}, wherein R^{a}, R^{b}, and R^{c} are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy. In a second subembodiment of embodiment 10, Ar¹ is phenyl, 4-fluorophenyl, 4-chlorophenyl, 4-bromophenyl, 3,4-dichlorophenyl, 2,4-difluorophenyl, 4-methoxyphenyl, 4-cyclopropoxyphenyl, 4-trifluoromethoxyphenyl, 3- or 4-CONH₂phenyl, or 4-cyanophenyl.

11. In embodiment 11, the compound of any one of embodiments 1 to 9 (and embodiments and subembodiments contained therein), or a pharmaceutically acceptable salt thereof, is wherein Ar¹ is heteroaryl, wherein said heteroaryl is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂. In first subembodiment of embodiment 11, R^{a}, R^{b}, and R^{c} are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy.

12. In embodiment 12, the compound of any one of embodiments 1 to 10 (and embodiments and subembodiments contained therein), or a pharmaceutically acceptable salt thereof, is wherein X¹ is NH.

13. In embodiment 13, the compound of any one of embodiments 1 to 10 (and embodiments and subembodiments contained therein), or a pharmaceutically acceptable salt thereof, is wherein X¹ is O.

14. In embodiment 14, the compound of any one of embodiments 1 to 13 (and embodiments and subembodiments contained therein), or a pharmaceutically acceptable salt thereof, is wherein R² and R³ are independently selected from hydrogen, methyl, methoxy, hydroxy, fluoro, chloro, trifluoromethyl, trifluoromethoxy, or cyano. In a first subembodiment of embodiment 14, wherein R² is hydrogen and R³ is selected from hydrogen, methyl, fluoro, chloro, trifluoromethyl, trifluoromethoxy, or cyano.

It is understood that the embodiments set forth above include combinations of one or more of embodiments and/or subembodiments listed therein. For example, the Ar¹ group listed in embodiment 9 and subembodiment therein, can independently combine with one or more of the embodiments 1-8, and 14 and/or subembodiments contained therein.

### General Synthetic Schemes

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) and (II) where X¹ is NH and other groups are as defined in the Summary can be prepared the method illustrated and described in Scheme 1 below.

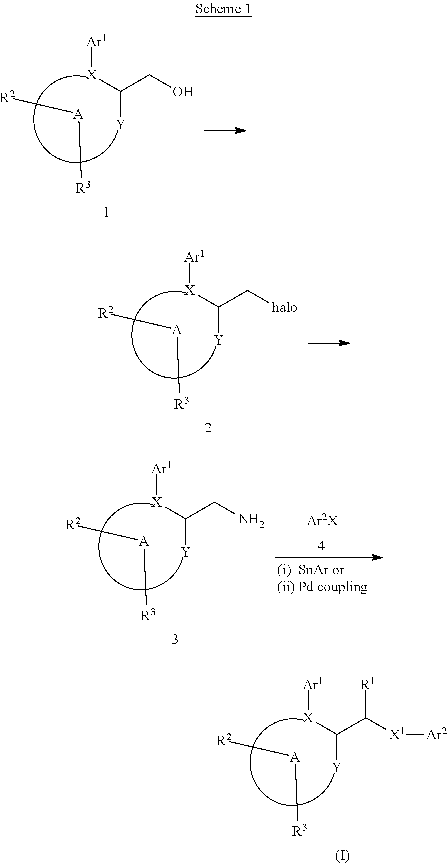

Reaction of an alcohol of formula 1 where Ar¹, ring A, and R² and R are as defined in the Summary with a halogenating agent such as sulfonyl chloride, oxalyl chloride (when halo is chloro) under suitable halogenating agent known in the art provides a compound of formula 2. Alcohols 2 can be prepared by method well known in the art. Some such methods are described in synthetic examples below. Compound 2 is converted to an amine of formula 3, either directly by reacting 2 with ammonia in an alcohol solvent e.g., methanol or indirectly by first converting 2 to a corresponding phthalimido derivative by reacting 2 with phthalimide salt such as potassium phthalimde, followed by hydrolysis of the phthalimide derivative by methods well known in the art.

Compounds of Formula (I) and (II) can be prepared by reacting an amine of formula 3 or it's salt with an aryl halide of formula 4 where Ar² is as defined in the Summary under S_{N}Ar reaction conditions i.e., in the presence of a base such as N-methylpyridine, diethylisopropylamine, pyridine, and the like, or under Palladium reaction conditions well known in the art.

Compounds of formula 4 are either commercially available or can be prepared by methods well known in the art.

Compounds of Formula (I) and (II) where X¹ is O and other groups are as defined in the Summary can be prepared by reacting a compound of formula 1 with an aryl halide of formula 4 under S_{N}Ar reaction conditions.

Alternatively, compounds of Formula (I) and (II) where X is NH and other groups are as defined in the Summary can be prepared the method illustrated and described in Scheme 2 below.

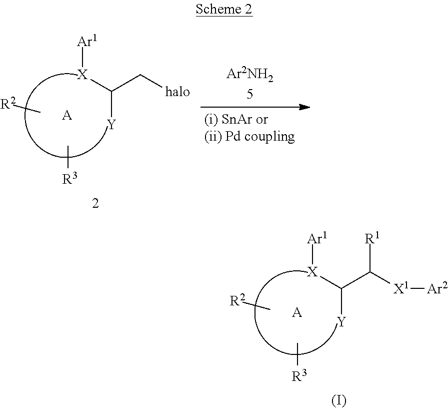

Compounds of Formula (I) and (II) can also be prepared by reacting compound 2 with an arylamine of formula 5 here Ar² is as defined in the Summary under S_{N}Ar or Palladium compound reaction conditions well known in the art.

### Assay

The ability of compounds of the disclosure to inhibit Polθ can be measured as described in Biological Example 1 below.

### Pharmaceutical Composition

The compounds of Formula (I), or a pharmaceutically acceptable salt thereof, provided herein may be in the form of compositions suitable for administration to a subject. In general, such compositions are pharmaceutical compositions comprising a compound of Formula (I) or (II) or a pharmaceutically acceptable salt thereof and one or more pharmaceutically acceptable or physiologically acceptable excipients. In certain embodiments, the compound of Formula (I) or (II), or a pharmaceutically acceptable salt thereof is present in a therapeutically effective amount. The pharmaceutical compositions may be used in the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., a compound of Formula (I) or (II), a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs.

Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound of Formula (I) or (II), or a salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a compound of Formula (I) or (II), or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound of Formula (I) or (II), or a salt thereof disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

A compound of Formula (I) or (II), or a salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

### Routes of Administration

Compounds of Formula (I) or (II), or a salt thereof and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to administer the compounds of Formula (I) or (II), or a salt thereof over a defined period of time. Particular embodiments of the present invention contemplate oral administration.

### Combination Therapy

The present invention contemplates the use of compounds of Formula (I) or (II), or a salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the compounds of Formula (I) or (II), or a salt thereof are administered or applied sequentially, e.g., where one agent is administered prior to one or more other agents. In other embodiments, the compounds of Formula (I) or (II), or a salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The compounds of Formula (I) or (II), or a salt thereof may be used in combination with at least one other (active) agent in any manner appropriate under the circumstances. In one embodiment, treatment with the at least one active agent and at least one compound of Formula (I) or (II), or a salt thereof is maintained over a period of time. In another embodiment, treatment with the at least one active agent is reduced or discontinued (e.g., when the subject is stable), while treatment with the compound of Formula (I) or (II), or a salt thereof is maintained at a constant dosing regimen. In a further embodiment, treatment with the at least one active agent is reduced or discontinued (e.g., when the subject is stable), while treatment with a compound of Formula (I) or (II), or a salt thereof is reduced (e.g., lower dose, less frequent dosing or shorter treatment regimen). In yet another embodiment, treatment with the at least one active agent is reduced or discontinued (e.g., when the subject is stable), and treatment with the compound of Formula (I) or (II), or a salt thereof is increased (e.g., higher dose, more frequent dosing or longer treatment regimen). In yet another embodiment, treatment with the at least one active agent is maintained and treatment with the compound of Formula (I) or (II), or a salt thereof is reduced or discontinued (e.g., lower dose, less frequent dosing or shorter treatment regimen). In yet another embodiment, treatment with the at least one active agent and treatment with the compound of Formula (I) or (II), or a salt thereof are reduced or discontinued (e.g., lower dose, less frequent dosing or shorter treatment regimen).

The present disclosure provides methods for treating cancer with a compound of Formula (I) or (II), or a salt thereof and at least one additional therapeutic or diagnostic agent.

In some embodiments, the compound of Formula (I) or (II), or a salt thereof is administered in combination with at least one additional therapeutic agent, selected from Temozolomide, Pemetrexed, Pegylated liposomal doxorubicin (Doxil), Eribulin (Halaven), Ixabepilone (Ixempra), Protein-bound paclitaxel (Abraxane), Oxaliplatin, Irinotecan, Venatoclax (bcl2 inhibitor), 5-azacytadine, Anti-CD20 therapeutics, such as Rituxan and obinutuzumab, Hormonal agents (anastrozole, exemestand, letrozole, zoladex, lupon eligard), CDK4/6 inhibitors, Palbociclib, Abemaciclib, CPI (Avelumab, Cemiplimab-rwlc, and Bevacizumab.

In certain embodiments, the present disclosure provides methods for treating cancer comprising administration of a compound of Formula (I) or (II), or a salt thereof described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Examples of signal transduction inhibitors (STIs) useful in methods described herein include, but are not limited to: (i) bcr/abl kinase inhibitors (e.g., GLEEVEC); (ii) epidermal growth factor (EGF) receptor inhibitors, including kinase inhibitors and antibodies; (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with one or more compounds of Formula (I) or (II), or a salt thereof described herein for the suppression of tumor growth in cancer patients.

In certain embodiments, the present disclosure provides methods for treating cancer comprising administration of a compound of Formula (I) or (II), or a salt thereof described herein in combination with a chemotherapeutic agents. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; PARP inhibitors such as olaparib, rucaparib, niraparib, talazoparib, veliparib, and pamiparib, DNA damage repair inhibitors such as inhibitors of ATM [such as AZ: (AZD1390) Astrazeneca's AZD0156, AZ31, AZ32; Kudos' KU-55933, KU-60019, and KU-59403; and Pfizer's CP-466722]; ATR [such as Astrazeneca's Ceralasertib (AZD6738); Repare's RP-3500; Vertex/EMD Serono's Berzosertib (VX-970/M6620); and EMD Serono's M4344; and DNA-PK (such as Astrazeneca's AZD7648; NU7441; NU7026; Kudos' KU-0060648; Vertex's VX-984; and EMD Serono's Nedisertib (M3814)] and Cyteir Therapeutics RAD51 inhibitor CYT-0851 and pharmaceutically acceptable salts, acids or derivatives of any of the above. In a particular embodiment, compounds of the present disclosure are coadministered with a cytostatic compound selected from the group consisting of cisplatin, doxorubicin, taxol, taxotere and mitomycin C. In a particular embodiment, the cytostatic compound is doxorubicin.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, enzalutamide, apalutamide, abiraterone acetate, leuprolide, and goserelin; and pharmaceutically acceptable salts, acids or derivatives of any of the above. In certain embodiments, combination therapy comprises administration of a hormone or related hormonal agent.

The present disclosure also contemplates the use of the compounds of Formula (I) or (II), or a salt thereof described herein in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGFβ, CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure contemplates the use of compounds of Formula (I) or (II), or a salt thereof described herein in combination with inhibitors of the aforementioned immune-checkpoint receptors and ligands, for example ipilimumab, abatacept, nivolumab, pembrolizumab, atezolizumab, nivolumab, and durvalumab.

Additional treatment modalities that may be used in combination with a compound of Formula (I) or (II), or a salt thereof disclosed herein include radiotherapy, a monoclonal antibody against a tumor antigen, a complex of a monoclonal antibody and toxin, a T-cell adjuvant, bone marrow transplant, or antigen presenting cells (e.g., dendritic cell therapy).

The present disclosure contemplates the use of compounds of Formula (I) or (II), or a salt thereof described herein for the treatment of glioblastoma either alone or in combination with radiation and/or temozolomide (TMZ), avastin or lomustine.

The present disclosure encompasses pharmaceutically acceptable salts, acids or derivatives of any of the above.

### Dosing

The compounds of Formula (I) or (II), or a salt thereof provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of Formula (I) or (II), or a salt thereof, as provided herein, may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the compounds of Formula (I) or (II), or a salt thereof disclosed herein may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the compound of Formula (I) or (II), or a salt thereof is contained in a “unit dosage form”. The phrase “unit dosage form” refers to physically discrete units, each unit containing a predetermined amount of the compound of Formula (I) or (II), or a salt thereof, either alone or in combination with one or more additional agents, sufficient to produce the desired effect. It will be appreciated that the parameters of a unit dosage form will depend on the particular agent and the effect to be achieved.

### Kits

The present invention also contemplates kits comprising a compound of Formula (I) or (II), or a salt thereof, and pharmaceutical compositions thereof. The kits are generally in the form of a physical structure housing various components, as described below, and may be utilized, for example, in practicing the methods described above.

A kit can include one or more of the compound of Formula (I) or (II), or a salt thereof disclosed herein (provided in, e.g., a sterile container), which may be in the form of a pharmaceutical composition suitable for administration to a subject. The compound of Formula (I) or (II), or a salt thereof can be provided in a form that is ready for use (e.g., a tablet or capsule) or in a form requiring, for example, reconstitution or dilution (e.g., a powder) prior to administration. When the compounds of Formula (I) or (II), or a salt thereof are in a form that needs to be reconstituted or diluted by a user, the kit may also include diluents (e.g., sterile water), buffers, pharmaceutically acceptable excipients, and the like, packaged with or separately from the compounds of Formula (I) or (II), for a salt thereof. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples and references (intermediates) are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: μg=microgram; μl or μL=microliter; mM=millimolar; μM=micromolar; THF=tetrahydrofuran; DIEA=diisopropylethylamine; EtOAc=ethyl acetate; NMP=N-methylpyridine, TFA=trifluoroacetic acid; DCM=dichloromethane; Cs₂CO₃=cesium carbonate; XPhos Pd G3=2-dicyclohexylphosphino-2′,4′,6′-triisopropyl-1,1′-biphenyl)[2-(2′-amino-1,1′-biphenyl)]palladium-(II) methanesulfonate; LiCl=lithium chloride; POC₃=phosphoryl chloride; PE=petroleum ether; DMSO=dimethylsulfoxide; HCl=hydrochloric acid; Na₂SO₄=sodium sulfate; DMF=dimethylformamide; NaOH=sodium hydroxide; K₂CO₃=potassium carbonate; MeCN=acetonitrile; BOC=tert-butoxycarbonyl; MTBE=methyl tert-butyl ether; MeOH=methanol; NaHCO₃=sodium bicarbonate; NaBH₃CN=sodium cyanoborohydride; EtOH=ethanol; PCl₅=phosphorus pentachloride; NH₄OAc=ammonium acetate; Et₂O=ether; HOAc=acetic acid; Ac₂O=acetic anhydride; i-PrOH=isopropanol; NCS=N-chlorosuccinimide; K₃PO₄=potassium phosphate; Pd(dtbpf)Cl₂=1,1′-bis(di-tert-butylphosphino)ferrocene]dichloropalladium(II); Zn(CN)₂=Zinc cyanide; Pd(PPh₃)₄=tetrakis(triphenylphosphine)palladium(O); Et₃N=triethylamine; CuCN=copper cyanide; t-BuONO=tert-butyl nitrite; HATU=1-[bis(dimethylamino)methylene]-1H-1,2,3-triazolo[4,5-b]pyridinium 3-oxid hexafluorophosphate; DBU=1,8-diazabicyclo(5.4.0)undec-7-ene; LiAlH₄=lithium aluminium hydride; NH₃=ammonia; H₂SO₄=sulfuric acid; H₂O₂=hydrogen peroxide;

### SYNTHETIC EXAMPLES

### General Procedures

### Procedure A

### Preparation of Aryl Alcohol

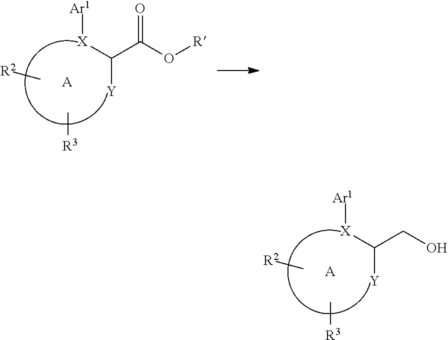

To a solution of ester (1 eq.) in THE (0.3M) was added LiAlH₄ (2 eq.) at 0° C. The mixture was stirred for 2 h at 0° C.

### Procedure B

### Preparation of Alkyl Chloride

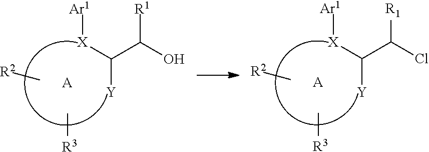

To a solution of alcohol (1 eq.) in DCM (0.5M) was added SOCl₂ (2 eq.) at room temperature under nitrogen. The mixture was stirred overnight at room temperature. The mixture was concentrated under reduced pressure and the residue was used in the next step without further purification.

### Procedure C

### Preparation of Alkyl Amine

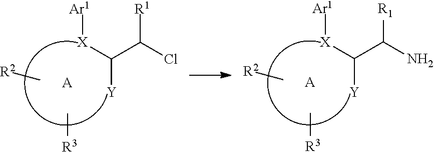

The alkyl chloride (1 eq.) was diluted with NH₃ in MeOH (8M, 200 eq.). The mixture was stirred overnight at 50° C. The mixture was concentrated under reduced pressure.

### Procedure D

### Preparation of Alkyl Phthalimide

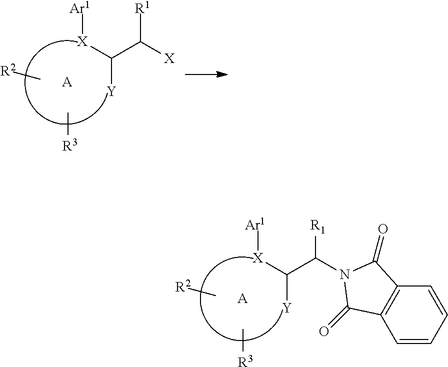

To a solution of alkyl halide (X is halo) (1 eq.) in DMF (0.4 M) was added potassium phthalimide (2 eq.). The mixture was stirred for 2 h at room temperature under nitrogen. The mixture was diluted with water and extracted with EtOAc. The combined organic layers were concentrated under reduced pressure.

### Procedure E

### Deprotection of Alkyl Phthalimide

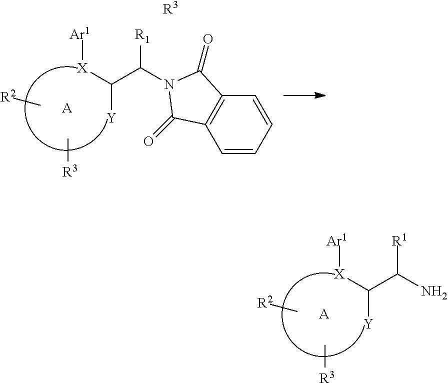

To a solution of 1,3-dione (1 eq.) in EtOH (0.25 M) was added hydrazine hydrate (2 eq.) at room temperature under nitrogen. The mixture was stirred for overnight at 60° C. under nitrogen.

### Procedure F

### N-Arylation Via S_{N}Ar

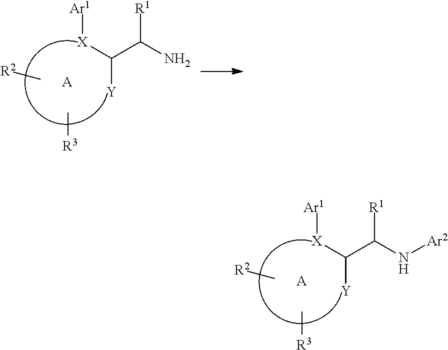

To a solution of amine (1 eq.) and arylchloride (1 eq.) in NMP (0.1 M) was added DIEA (2 eq.) at room temperature under nitrogen. The mixture was stirred for overnight at 50° C. under nitrogen.

### Procedure G

### N-Arylation Via Palladium Coupling

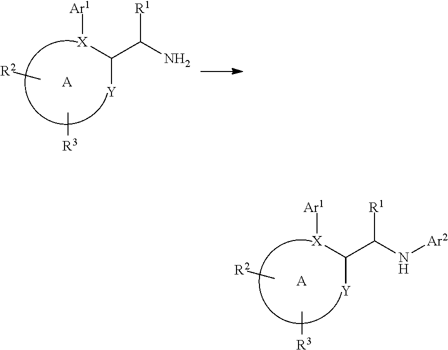

To a solution of alkyl amine (1 eq.) in dioxane (0.4M) was added aryl chloride (2 eq.), Cs₂CO₃ (2 eq.), and [(2-Di-tert-butylphosphino-2′,4′,6′-triisopropyl-1,1-biphenyl)-2-(2′-amino-1,1′-biphenyl)] palladium(II) methanesulfonate (tBuXPhos Pd G3) (0.15 eq.). The mixture was stirred for 12 h at 100° C. The mixture was diluted with water and extracted EtOAc (2×). The combined organic layers were concentrated under reduced pressure.

### Intermediate A

### Synthesis of 2-chloro-4,6-bis(trifluoromethyl)pyridine-3-carbonitrile

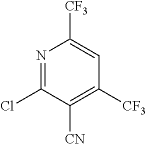 [SMILES: N#Cc1c(C(F)(F)F)cc(C(F)(F)F)nc1Cl]

To a solution of 1,1,1,5,5,5-hexafluoropentane-2,4-dione (25 g, 120 mmol) in sulfolane (50 mL) was added 2-cyanoacetamide (10 g, 120 mmol). The mixture was stirred overnight at 150° C. and then diluted with EtOAc and washed with LiCl. The organic layer was dried over Na₂SO₄ and concentrated under reduced pressure to give a yellow solid. The solid was dissolved in POCl₃ (36 g, 236 mmol) and after adding Et₃N (9.6 g, 94 mmol) the mixture was stirred overnight at 125° C. and then quenched with ice water. The mixture was extracted with EtOAc and the combined organic layers were washed with water and concentrated under reduced pressure. The residue was purified using silica gel chromatography (eluent: 1% EtOAc in PE) to afford 4.5 g (35% yield) of 2-chloro-4,6-bis(trifluoromethyl)pyridine-3-carbonitrile as light-yellow oil. ¹H NMR (300 MHz; DMSO-d₆): δ 8.64 (s, 1H) ppm.

### Example 1

### Synthesis of 5-chloro-4,6-dimethyl-2-((1-phenyl-1H-imidazol-2-yl)methoxy)nicotinonitrile

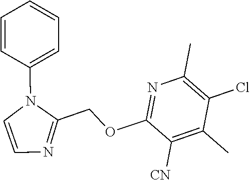

### Step 1: Preparation of methyl 1-phenylimidazole-2-carboxylate

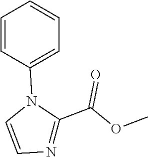

To a solution of ethyl 1H-imidazole-2-carboxylate (5.0 g, 35.7 mmol) in MeOH (100 mL) was added phenyl boronic acid (8.7 g, 71.4 mmol), pyridine (8.47 g, 107.0 mmol) and Cu(OAc)₂ (9.7 g, 53.5 mmol). The mixture was stirred overnight at room temperature open to air and then filtered. The filtrate and concentrated under reduced pressure and the residue was purified using silica gel chromatography (eluent: 33% EtOAc in hexanes) to give 650 mg (9% yield) of methyl 1-phenylimidazole-2-carboxylate as a white solid.

### Step 2: Preparation of (1-phenylimidazol-2-yl)methanol

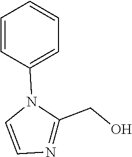 [SMILES: OCc1nccn1-c1ccccc1]

The title compound was prepared using General Procedure A employing methyl 1-phenylimidazole-2-carboxylate. The mixture was quenched with sat. NH₄Cl and extracted with EtOAc and the combine organic layers were concentrated. The residue was purified using silica gel chromatography (eluent: 1% MeOH in DCM) to afford 320 mg (68% yield) of the title compound as a white solid.

### Step 3: Preparation of 5-chloro-4,6-dimethyl-2-((1-phenyl-1H-imidazol-2-yl)methoxy)-nicotinonitrile

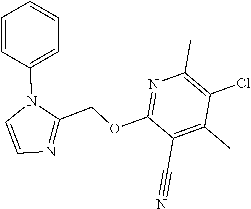 [SMILES: Cc1nc(OCc2nccn2-c2ccccc2)c(C#N)c(C)c1Cl]

To a solution of (1-phenyl-1H-imidazol-2-yl)methanol (150 mg, 0.86 mmol) in 2-methoxyethyl ether (1.5 mL) was added a solution of 2,5-dichloro-4,6-dimethylpyridine-3-carbonitrile (173 mg, 0.86 mmol) in 2-methoxyethyl ether (1.5 mL). K₂CO₃ (238 mg, 1.7 mmol) was added and the mixture was stirred overnight at 130° C. The mixture was diluted with water and extracted with EtOAc. The combined organic layers were concentrated under reduced pressure and the residue was purified by Prep-TLC (50:1, DCM:MeOH) to afford 63 mg of the title compound as a yellow solid. ¹H NMR (400 MHz; CDCl₃): δ2.36-2.55 (m, 6H), 5.50 (s, 2H), 7.18 (d, 1H), 7.26-7.33 (m, 1H), 7.46-7.54 (m, 5H) ppm. m/z 339 (M+H⁺).

### Example 2

### Synthesis of 5-chloro-4,6-dimethyl-2-((1-phenyl-1H-imidazol-2-yl)methylamino)nicotinonitrile

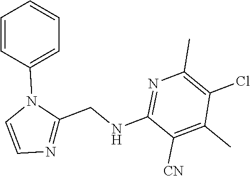 [SMILES: Cc1nc(NCc2nccn2-c2ccccc2)c(C#N)c(C)c1Cl]

### Step 1: Preparation of 2-((1-phenyl-1H-imidazol-2-yl)methyl)isoindoline-1,3-dione

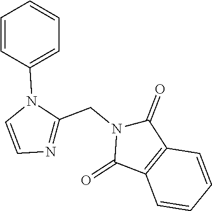 [SMILES: O=C1c2ccccc2C(=O)N1Cc1nccn1-c1ccccc1]

To a solution of (1-phenyl-1H-imidazol-2-yl)methanol (250 mg, 1.4 mmol, Example 1, Step 2) in THE (4 mL) was added 2,3-dihydro-1H-isoindole-1,3-dione (253 mg, 1.7 mmol), DIAD (580 mg, 2.9 mmol) and PPh₃ (753 mg, 2.9 mmol) in portions at room temperature. The mixture was stirred for overnight at room temperature under nitrogen and then concentrated under reduced pressure. The residue was purified using silica gel chromatography (eluent: 2% EtOAc in PE) to afford the title compound (150 mg, 34% yield) as a white solid.

### Step 2: Preparation of (1-phenyl-1H-imidazol-2-yl)methanamine

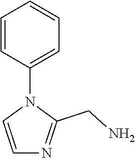 [SMILES: NCc1nccn1-c1ccccc1]

The title compound was prepared using General Procedure E employing 2-((1-phenyl-1H-imidazol-2-yl)methyl)isoindoline-1,3-dione. The mixture was cooled to room temperature and filtered and the solid was washed with EtOH. The filtrate was concentrated under reduced pressure and the residue was purified by reverse phase chromatography (column, C₁₈ silica gel; mobile phase, 10-50% MeOH in water) to afford the title compound (45 mg, 53% yield) as a white solid.

### Step 3: Preparation of 5-chloro-4,6-dimethyl-2-((1-phenyl-1H-imidazol-2-yl)methylamino-)nicotinonitrile

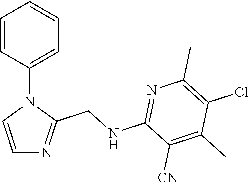 [SMILES: Cc1nc(NCc2nccn2-c2ccccc2)c(C#N)c(C)c1Cl]

The title compound was prepared using General Procedure F employing (1-phenyl-1H-imidazol-2-yl)methanamine and 2,5-dichloro-4,6-dimethylnicotinonitrile. The mixture was diluted with water and extracted with EtOAc and the combined organic layers were washed with water. The mixture was concentrated under reduced pressure and the residue was purified by Prep-TLC (150:1, DCM:MeOH) to afford the title compound (2.4 mg, 3% yield) as a white solid. ¹H NMR (300 MHz; CDCl₃): δ 7.46-7.52 (m, 3H), 7.32-7.39 (m, 2H), 7.09-7.17 (m, 2H), 5.95 (s, 1H), 4.71 (d, 2H), 2.44 (s, 3H), 2.39 (s, 3H) ppm. m/z 338 (M+H⁺).

### Example 3

### Synthesis of 5-chloro-2-(((1-(4-fluorophenyl)-1H-imidazol-2-yl)methyl)amino)-4,6-dimethylnicotinonitrile

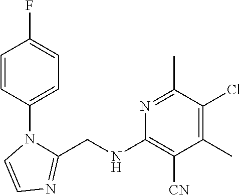 [SMILES: Cc1nc(NCc2nccn2-c2ccc(F)cc2)c(C#N)c(C)c1Cl]

### Step 1: Preparation of ethyl 1-(4-fluorophenyl)-1H-imidazole-2-carboxylate

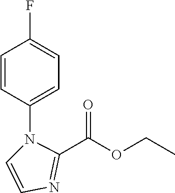 [SMILES: CCOC(=O)c1nccn1-c1ccc(F)cc1]

To a solution of ethyl 1H-imidazole-2-carboxylate (10 g, 71.3 mmol) in DCM (100 mL) Cu(OAc)₂ (19.4 g, 107.0 mmol), pyridine (11.3 g, 142.7 mmol) and (4-fluorophenyl)boronic acid (19.3 g, 142.7 mmol) were added at room temperature. The mixture was stirred overnight at room temperature open to air. The mixture was filtered and the solid was washed with EtOAc and then concentrated under reduced pressure. The residue was purified using silica gel chromatography (eluent: 33% EtOAc in PE) to afford the title compound (8.3 g) as a white solid.

### Step 2: Preparation of [1-(4-fluorophenyl)-1H-imidazol-2-yl]methanol

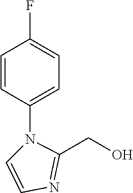 [SMILES: OCc1nccn1-c1ccc(F)cc1]

The title compound was prepared using General Procedure A employing ethyl 1-(4-fluorophenyl)-1H-imidazole-2-carboxylate. The mixture was quenched with sat. NHCl and extracted with EtOAc. The combined organic layers were concentrated under reduced pressure and the residue was purified using silica gel chromatography (eluent: 3% MeOH in DCM) to afford the title compound (5.2 g) as a white solid.

### Step 3: Preparation of 2-(chloromethyl)-1-(4-fluorophenyl)-1H-imidazole

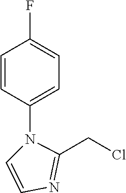 [SMILES: Fc1ccc(-n2ccnc2CCl)cc1]

The title compound was prepared using General Procedure B employing [1-(4-fluorophenyl)-1H-imidazol-2-yl]methanol to afford the title compound.

### Step 4: Preparation of 1-[1-(4-fluorophenyl)-1H-imidazol-2-yl]methanamine

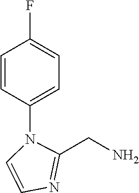 [SMILES: NCc1nccn1-c1ccc(F)cc1]

The title compound was prepared using General Procedure C employing 2-(chloromethyl)-1-(4-fluorophenyl)-1H-imidazole. The residue was purified using silica gel chromatography (eluent: 17% MeOH in DCM) to afford the title compound (2.7 g) as brown oil.

### Step 5: Preparation of 5-chloro-2-(((1-(4-fluorophenyl)-1H-imidazol-2-yl)methyl)-amino)-4,6-dimethylnicotinonitrile

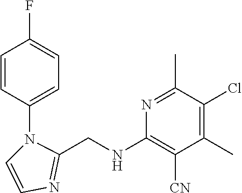 [SMILES: Cc1nc(NCc2nccn2-c2ccc(F)cc2)c(C#N)c(C)c1Cl]

The title compound was prepared using General Procedure G using 1-[1-(4-fluorophenyl)-1H-imidazol-2-yl]methanamine and (2,5-dichloro-4,6-dimethylpyridine-3-carbonitrile). The residue was purified by Prep-TLC (50:1, DCM:MeOH) to afford 41 mg of the title compound as a white solid. ¹H NMR (400 MHz; DMSO-d₆): δ 7.52-7.55 (m, 2H), 7.31-7.37 (m, 4H), 6.98 (d, 1H), 4.54 (d, 2H), 2.38 (s, 3H), 2.30 (s, 3H) ppm. m/z 356 (M+H⁺).

### Example 4

### Synthesis of 3-chloro-N-((1-(4-fluorophenyl)-1H-imidazol-2-yl)methyl)-5-(trifluoromethyl)benzenamine

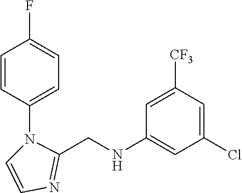 [SMILES: Fc1ccc(-n2ccnc2CNc2cc(Cl)cc(C(F)(F)F)c2)cc1]

The title compound was prepared using General Procedure G 1-[1-(4-fluorophenyl)-1H-imidazol-2-yl]methanamine (Example 3, Step 4) and 1-bromo-3-chloro-5-(trifluoromethyl)-benzene. The residue was purified by Prep-TLC (10:1, DCM:MeOH) to afford 20.9 mg of the title compound as a white solid. ¹H NMR (400 MHz; DMSO-d₆): δ 7.58-7.54 (m, 2H), 7.46 (s, 1H), 7.40-7.36 (m, 2H), 7.16 (s, 1H), 6.87-6.82 (m, 4H), 4.36 (d, 2H). m/z 370 (M+H⁺).

### Example 5

### Synthesis of 2-([[1-(4-fluorophenyl)-1H-imidazol-2-yl]methyl]amino)-4,6-bis trifluoromethyl)pyridine-3-carbonitrile

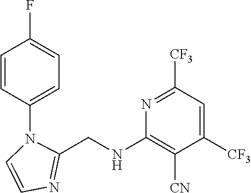 [SMILES: N#Cc1c(C(F)(F)F)cc(C(F)(F)F)nc1NCc1nccn1-c1ccc(F)cc1]

The title compound was prepared using General Procedure F employing 1-[1-(4-fluorophenyl)-1H-imidazol-2-yl]methanamine and 2-chloro-4,6-bis(trifluoromethyl)pyridine-3-carbonitrile (Intermediate A). The mixture was stirred for 2 h at 100° C. and then diluted with water and extracted with EtOAc. The organic layer was concentrated under reduced pressure and the residue was purified by Prep-TLC (1:1, PE:EtOAc) to afford the title compound as a white solid. ¹H NMR (300 MHz; DMSO-d₆): δ 8.61-8.65 (m, 1H), 7.50-7.56 (m, 2H), 7.45 (s, 1H), 7.29-7.40 (m, 3H), 7.00 (s, 1H), 4.65 (d, 2H) ppm. m/z 430 (M+H⁺).

### Example 6

### Synthesis of 2-([[1-(4-methoxyphenyl)-1H-imidazol-2-yl]methyl]amino)-4,6-bis(trifluoromethyl)pyridine-3-carbonitrile

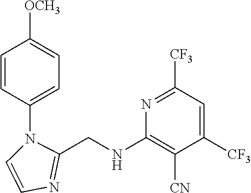 [SMILES: COc1ccc(-n2ccnc2CNc2nc(C(F)(F)F)cc(C(F)(F)F)c2C#N)cc1]

### Step 1: Preparation of ethyl 1-(4-methoxyphenyl)-1H-imidazole-2-carboxylate

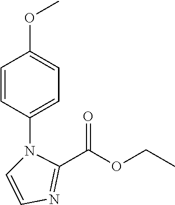 [SMILES: CCOC(=O)c1nccn1-c1ccc(OC)cc1]

Proceeding analogously as described in Example 1, Step 1 but substituting phenyl boronic acid with (4-methoxyphenyl)boronic acid provided the title as a white solid.

### Step 2: Preparation of [1-(4-methoxyphenyl)-1H-imidazol-2-yl]methanol

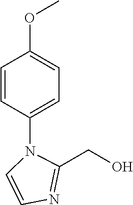

Proceeding analogously as described in Example 1, Step 2 but substituting methyl 1-phenylimidazole-2-carboxylate with (4-methoxyphenyl)-1H-imidazole-2-carboxylate provided the title compound as a white solid.

### Step 3: Preparation of 2-(chloromethyl)-1-(4-methoxyphenyl)-1H-imidazole

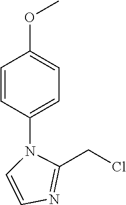

The title compound was prepared using General Procedure B employing [1-(4-methoxyphenyl)-1H-imidazol-2-yl]methanol. The mixture was stirred for 4 h at room temperature.

### Step 4: Preparation of 1-[1-(4-methoxyphenyl)-1H-imidazol-2-yl]methanamine

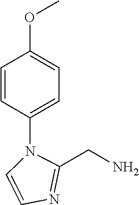

The title compound was prepared using General Procedure C employing 2-(chloro-methyl)-1-(4-methoxyphenyl)-1H-imidazole. The residue was purified using silica gel chromatography (eluent: 16% MeOH in DCM) to afford the title compound as a brown oil.

### Step 5: Preparation of 2-([[1-(4-methoxyphenyl)-1H-imidazol-2-yl]methyl]amino)-4,6-bis(trifluoromethyl)pyridine-3-carbonitrile

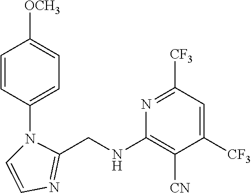 [SMILES: COc1ccc(-n2ccnc2CNc2nc(C(F)(F)F)cc(C(F)(F)F)c2C#N)cc1]

The title compound was prepared using General Procedure F employing 1-[1-(4-methoxyphenyl)-1H-imidazol-2-yl]methanamine and 2-chloro-4,6-bis(trifluoromethyl) pyridine-3-carbonitrile (Intermediate A). The mixture was stirred overnight at room temperature under nitrogen, diluted with water and extracted with EtOAc. The combined organic layers were washed with water, dried over Na₂SO₄ and concentrated under reduced pressure. The residue was purified by Prep-TLC (20:1, DCM:MeOH) to afford the title compound as a yellow solid. ¹H NMR (300 MHz; DMSO-d₆): δ 8.69 (t, 1H), 7.40-7.29 (m, 4H), 7.01-6.96 (m, 3H), 4.64 (d, 2H), 3.80 (s, 3H) ppm. m/z 442 (M+H⁺).

### Example 7

### Synthesis of 2-[([1-[(4-fluorophenyl)methyl]-1H-imidazol-2-yl]methyl)amino]-4,6-bis(trifluoromethyl)pyridine-3-carbonitrile

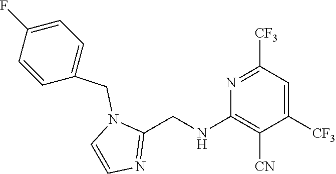 [SMILES: N#Cc1c(C(F)(F)F)cc(C(F)(F)F)nc1NCc1nccn1Cc1ccc(F)cc1]

### Step 1: Preparation of ethyl 1-[(4-fluorophenyl)methyl]-1H-imidazole-2-carboxylate

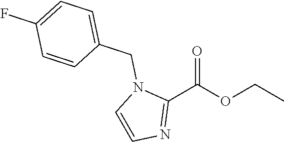 [SMILES: CCOC(=O)c1nccn1Cc1ccc(F)cc1]

To a solution of ethyl 1H-imidazole-2-carboxylate (5 g, 35.7 mmol) in DMF (50 mL) was added 1-(bromomethyl)-4-fluorobenzene (6.7 g, 36 mmol) and K₂CO₃ (9.9 g, 71 mmol) and the mixture was stirred for 2 h at room temperature under nitrogen. The mixture was diluted with water and extracted with Et₂O and the combined organic layers were concentrated under reduced pressure. The residue was purified using silica gel chromatography (eluent: 15% EtOAc in PE) to afford the title compound (5.3 g, 48% yield) as an orange oil.

### Step 2: Preparation of [1-[(4-fluorophenyl)methyl]-1H-imidazol-2-yl]methanol

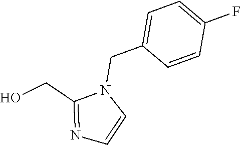 [SMILES: OCc1nccn1Cc1ccc(F)cc1]

Proceeding analogously as described in Example 3, Step 2, but substituting ethyl 1-(4-fluorophenyl)-1H-imidazole-2-carboxylate with ethyl 1-[(4-fluorophenyl)methyl]-1H-imidazole-2-carboxylate provided the title compound as an orange solid.

### Step 3: Preparation of 2-(chloromethyl)-1-[(4-fluorophenyl)methyl]-1H-imidazole

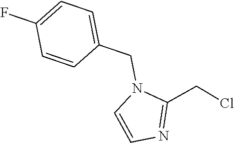 [SMILES: Fc1ccc(Cn2ccnc2CCl)cc1]

The title compound was prepared using General Procedure B employing [1-[(4-fluorophenyl) methyl]-1H-imidazol-2-yl]methanol. The mixture was stirred for 4 h at room temperature under nitrogen.

### Step 4: Preparation of 1-[1-[(4-fluorophenyl)methyl]-1H-imidazol-2-yl]methanamine

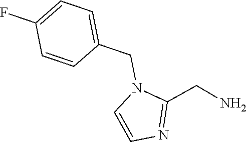 [SMILES: NCc1nccn1Cc1ccc(F)cc1]

The title compound was prepared using General Procedure C employing 2-(chloro-methyl)-1-[(4-fluorophenyl)methyl]-1H-imidazole. The residue was purified using silica gel chromatography (eluent: 2% MeOH in DCM) to afford the title compound as a dark brown oil.

### Step 5: Preparation of 2-[([1-[(4-fluorophenyl)methyl]-1H-imidazol-2-yl]methyl)amino]-4,6-bis(trifluoromethyl)pyridine-3-carbonitrile

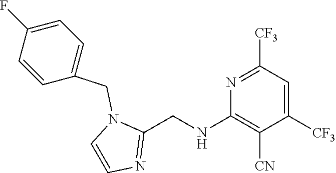 [SMILES: N#Cc1c(C(F)(F)F)cc(C(F)(F)F)nc1NCc1nccn1Cc1ccc(F)cc1]

Proceeding as described in Example 2, Step 3 but substituting (1-phenyl-1H-imidazol-2-yl)methanamine with 1-[1-[(4-fluorophenyl)methyl]-1H-imidazol-2-yl]methanamine and 2,5-dichloro-4,6-dimethylnicotinonitrile with 2-chloro-4,6-bis(trifluoromethyl)pyridine-3-carbonitrile (Intermediate A), followed by purification by Prep-TLC (20:1, DCM:MeOH) afforded the title compound as a white solid. ¹H NMR (300 MHz; DMSO-d₆): δ 8.50-8.59 (m, 1H), 7.38 (s, 1H), 7.21 (d, 1H), 7.01-7.10 (m, 2H), 6.97-6.99 (m, 2H), 6.88 (d, 1H), 5.27 (s, 2H), 4.65 (d, 2H) ppm. m/z 444 (M+H⁺).

### Example 8

### Synthesis of 2-[[1-(4-fluorophenyl)-1H-imidazol-2-yl]methoxy]-4,6-bis(trifluoromethyl)-pyridine-3-carbonitrile

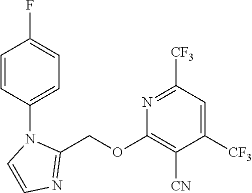 [SMILES: N#Cc1c(C(F)(F)F)cc(C(F)(F)F)nc1OCc1nccn1-c1ccc(F)cc1]

To a solution of [1-(4-fluorophenyl)-1H-imidazol-2-yl]methanol (70 mg, 0.36 mmol, Example 3, Step 2) in MeCN (2 mL) was added 2-chloro-4,6-bis(trifluoromethyl)pyridine-3-carbonitrile (100 mg, 0.36 mmol, Intermediate A) and K₂CO₃ (100 mg, 0.73 mmol). The mixture was stirred for 12 h at 90° C. and then filtered and the filtrate was concentrated under reduced pressure. The residue was purified by Prep-TLC (100:1, DCM:MeOH) to afford the title compound (43 mg) as a yellow solid. ¹H NMR (400 MHz; DMSO-d₆): δ 8.10 (s, 1H), 7.54-7.59 (m, 3H), 7.30-7.36 (m, 2H), 7.14 (d, 1H), 5.59 (s, 2H) ppm. m/z 431 (M+H⁺).

### Example 9

### Synthesis of 2-([[1-(4-fluorophenyl)-4-(trifluoromethyl)imidazol-2-yl]methyl]amino)-4,6-bis(trifluoromethyl)benzonitrile

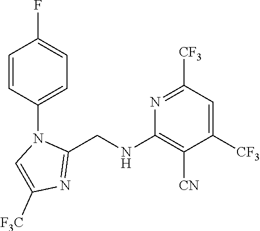 [SMILES: N#Cc1c(C(F)(F)F)cc(C(F)(F)F)nc1NCc1nc(C(F)(F)F)cn1-c1ccc(F)cc1]

### Step 1: Preparation of 2-[(benzyloxy)methyl]-4-(trifluoromethyl)-1H-imidazole

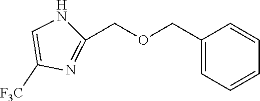 [SMILES: FC(F)(F)c1c[nH]c(COCc2ccccc2)n1]

To a solution of 3,3-dibromo-1,1,1-trifluoropropan-2-one (10 g, 37 mmol) in water (22.5 mL) was added NaOAc (55 g, 67 mmol). The mixture was stirred for 1 h at 100° C. under nitrogen and then a solution of 2-(benzyloxy)acetaldehyde (5.1 g, 33.7 mmol) in MeOH (155 mL) was added. A solution of ammonium hydroxide (38.4 mL) was added dropwise and the mixture was stirred overnight at room temperature. The mixture was diluted with water and extracted with EtOAc and the combined organic layers were concentrated under reduced pressure. The residue was purified using silica gel chromatography (eluent: 9% EtOAc in hexanes) to afford 3 g of the title compound (30% yield) as an off-white solid.

### Step 2: Preparation of 2-(benzyloxymethyl)-1-(4-fluorophenyl)-4-(trifluoromethyl)-1H-imidazole

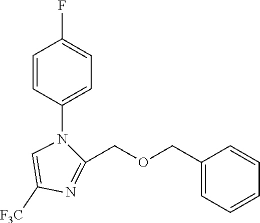 [SMILES: Fc1ccc(-n2cc(C(F)(F)F)nc2COCc2ccccc2)cc1]

To a solution of 2-[(benzyloxy)methyl]-4-(trifluoromethyl)-1H-imidazole (2.0 g, 7.8 mmol) in DCM (20 mL) was added pyridine (1.2 g, 15.6 mmol), 4-fluorophenylboronic acid (2.2 g, 15.6 mmol), and Cu(OAc)₂ (2.1 g, 11.7 mmol) and the mixture was stirred overnight at room temperature open to air. The mixture was filtered and the filtrate was concentrated under reduced pressure and the residue was purified by reverse chromatography (column, C₁₈ silica gel; mobile phase, 10-50% MeOH in water to afford 1.1 g (37% yield) of the title compound yellow oil.

### Step 3: Preparation of [1-(4-fluorophenyl)-4-(trifluoromethyl)imidazol-2-yl]methanol

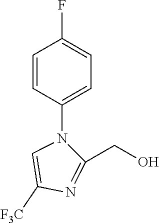 [SMILES: OCc1nc(C(F)(F)F)cn1-c1ccc(F)cc1]

To a solution of 2-[(benzyloxy)methyl]-1-(4-fluorophenyl)-4-(trifluoromethyl)imidazole (1.0 g, 2.9 mmol) in MeOH (250 mL) was added 10% Pd/C (500 mg) and the mixture was stirred for 2 h at room temperature under hydrogen. The mixture was filtered and solids were washed with MeOH. The filtrate was concentrated under reduced pressure to afford 770 mg (98% yield) of the title compound as a white solid which was used without further purification.

### Step 4: Preparation of 2-(chloromethyl)-1-(4-fluorophenyl)-4-(trifluoromethyl)imidazole

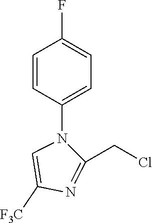 [SMILES: Fc1ccc(-n2cc(C(F)(F)F)nc2CCl)cc1]

The title compound was prepared using General Procedure B employing [1-(4-fluorophenyl)-4-(trifluoromethyl)imidazol-2-yl]methanol. The mixture was stirred for 2 h at room temperature.

### Step 5: Preparation of 2-[[1-(4-fluorophenyl)-4-(trifluoromethyl)imidazol-2-yl]methyl]isoindole-1,3-dione

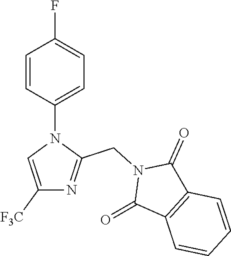 [SMILES: O=C1c2ccccc2C(=O)N1Cc1nc(C(F)(F)F)cn1-c1ccc(F)cc1]

The title compound was prepared using General Procedure D employing 2-(chloro-methyl)-1-(4-fluorophenyl)-4-(trifluoromethyl)imidazole. The residue was purified using silica gel chromatography (eluent: 33% EtOAc in hexanes) to afford 1.1 g (97% yield) of the title compound as a white solid.

### Step 6: Preparation of 1-[1-(4-fluorophenyl)-4-(trifluoromethyl)imidazol-2-yl]methanamine

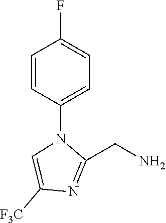 [SMILES: NCc1nc(C(F)(F)F)cn1-c1ccc(F)cc1]

The title compound was prepared using General Procedure E employing 2-[[1-(4-fluorophenyl)-4-(trifluoromethyl)imidazol-2-yl]methyl]isoindole-1,3-dione. After stirring overnight at 50° C., the mixture was filtered and the filtrate was concentrated under reduced pressure. The residue was purified by Prep-TLC (20:1, DCM:MeOH) to afford 630 mg (81% yield) of the title compound as colorless oil.

### Step 7: Preparation of 2-([[1-(4-fluorophenyl)-4-(trifluoromethyl)imidazol-2-yl]methyl]amino)-4,6-bis(trifluoromethyl)benzonitrile

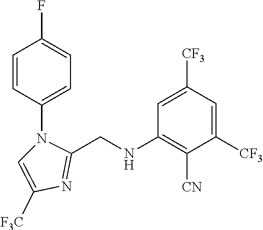 [SMILES: N#Cc1c(NCc2nc(C(F)(F)F)cn2-c2ccc(F)cc2)cc(C(F)(F)F)cc1C(F)(F)F]

The title compound was prepared using General Procedure F employing 2-chloro-4,6-bis(trifluoromethyl)pyridine-3-carbonitrile (Intermediate A) and 1-[1-(4-fluorophenyl)-4-(trifluoromethyl)imidazol-2-yl]methanamine. The mixture was stirred at 50° C. for 3 hours and then diluted with EtOAc and washed with LiCl. The organic layer was concentrated under reduced pressure and the residue was purified by Prep-TLC (150:1, DCM:MeOH) to afford the title compound as a white solid. ¹H NMR (400 MHz; DMSO-d₆): δ 8.66-8.69 (m, 1H), 8.05 (d, 1H), 7.53-7.56 (m, 2H), 7.40 (s, 1H), 7.28-7.30 (m, 2H), 4.68 (d, 2H) ppm. m/z 498 (M+H⁺).

### Example 10

### Synthesis of 2-((3-(4-fluorophenyl)pyrazin-2-yl)methylamino)-4,6-bis(trifluoromethyl)-nicotinonitrile

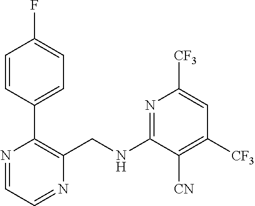 [SMILES: N#Cc1c(C(F)(F)F)cc(C(F)(F)F)nc1NCc1nccnc1-c1ccc(F)cc1]

### Step 1: Preparation of methyl 3-(4-fluorophenyl)pyrazine-2-carboxylate

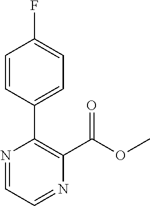 [SMILES: COC(=O)c1nccnc1-c1ccc(F)cc1]

To a solution of methyl 3-bromopyrazine-2-carboxylate (5.0 g, 23 mmol) in dioxane (50 mL) was added (4-fluorophenyl)boronic acid (6.5 g, 46 mmol), K₂CO₃ (6.4 g, 46 mmol) and Pd(dppf)Cl₂ (1.79 g, 2.3 mmol). The mixture was stirred for 1 h at 80° C. under nitrogen and then diluted with water and extracted with EtOAc. The combined organic layers were concentrated under reduced pressure and the residue was purified using silica gel chromatography (eluent: 9% EtOAc in PE) to afford the title compound (5.4 g, 99% yield) as a white solid.

### Step 2: Preparation of (3-(4-fluorophenyl)pyrazin-2-yl)methanol

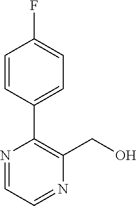 [SMILES: OCc1nccnc1-c1ccc(F)cc1]

To a solution of methyl 3-(4-fluorophenyl)pyrazine-2-carboxylate (5.0 g, 21.5 mmol) in DCM (50 mL) was added DIBAL-H (43.1 mL, 1M in n-hexane) at −78° C. The mixture was stirred for 2 h at −78° C. under nitrogen and then quenched by sat. NH₄Cl. The mixture was extracted with DCM and the combined organic layers were concentrated under reduced pressure. The residue was purified using silica gel chromatography (eluent: 2% MeOH in DCM) to afford 620 mg (14% yield) of the title compound as a white solid.

### Step 3: Preparation of 2-(chloromethyl)-3-(4-fluorophenyl)pyrazine

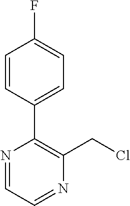 [SMILES: Fc1ccc(-c2nccnc2CCl)cc1]

The title compound was prepared using General Procedure B employing (3-(4-fluorophenyl)pyrazin-2-yl)methanol. The mixture was stirred for 2 h at room temperature.

### Step 4: Preparation of 2-((3-(4-fluorophenyl)pyrazin-2-yl)methyl)isoindoline-1,3-dione

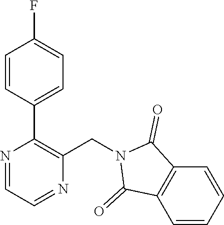 [SMILES: O=C1c2ccccc2C(=O)N1Cc1nccnc1-c1ccc(F)cc1]

The title compound was prepared using General Procedure D employing 2-(chloromethyl)-3-(4-fluorophenyl)pyrazine. The mixture was stirred overnight at room temperature. The mixture was diluted with water and extracted with EtOAc and the combined organic layers were concentrated under reduced pressure. The residue was purified using silica gel chromatography (eluent: 0.5% MeOH in DCM) to afford the title compound as a white solid.

### Step 5: Preparation of (3-(4-fluorophenyl)pyrazin-2-yl)methanamine

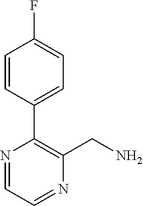 [SMILES: NCc1nccnc1-c1ccc(F)cc1]

The title compound was prepared using General Procedure E employing 2-((3-(4-fluorophenyl)pyrazin-2-yl)methyl)isoindoline-1,3-dione and hydrazine hydrate (5 eq.). The mixture was stirred for 2 h at 50° C. The mixture was filtered and the filtrate was concentrated to afford the title compound as a white solid, which was used without further purification.

### Step 6. Preparation of 2-((3-(4-fluorophenyl)pyrazin-2-yl)methylamino)-4,6-bis(trifluoromethyl)nicotinonitrile

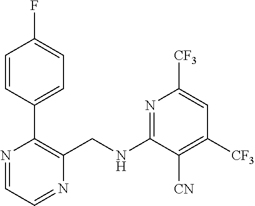 [SMILES: N#Cc1c(C(F)(F)F)cc(C(F)(F)F)nc1NCc1nccnc1-c1ccc(F)cc1]

The title compound was prepared using General Procedure E employing (3-(4-fluorophenyl)pyrazin-2-yl)methanamine and 2-chloro-4,6-bis(trifluoromethyl)pyridine-3-carbonitrile (Intermediate A). The mixture was diluted EtOAc and washed with LiCl. The organic layer was concentrated under reduced pressure and the residue was purified by Prep-TLC (5:1, PE:EtOAc) to afford 124 mg (57% yield) of the title compound as a light-yellow solid. ¹H NMR (300 MHz; DMSO-d₆): δ 8.71-8.74 (m, 1H), 8.56-8.60 (m, 2H), 7.65-7.69 (m, 2H), 7.29-7.36 (m, 3H), 4.82 (d, 2H) ppm. m/z 442 (M+H⁺).

### Example 11

### Synthesis of 2-((5-(4-fluorophenyl)pyrimidin-4-yl)methylamino)-4,6-bis(trifluoromethyl)nicotinonitrile

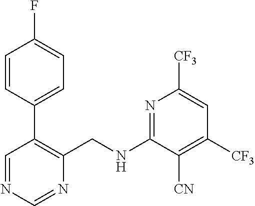 [SMILES: N#Cc1c(C(F)(F)F)cc(C(F)(F)F)nc1NCc1ncncc1-c1ccc(F)cc1]

### Step 1: Preparation of 5-(4-fluorophenyl)-4-methylpyrimidine

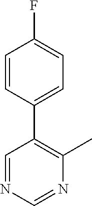

Proceeding analogously as described in Example 10, Step 1 but substituting methyl 3-bromopyrazine-2-carboxylate with 5-bromo-4-methylpyrimidine (2.0 g, 11.6 mmol) afforded 2.1 g (97% yield) of the title compound as a white solid.

### Step 2: Preparation of 4-(bromomethyl)-5-(4-fluorophenyl)pyrimidine

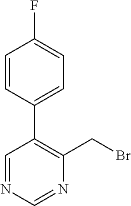 [SMILES: Fc1ccc(-c2cncnc2CBr)cc1]

To a solution of 5-(4-fluorophenyl)-4-methylpyrimidine (2.1 g, 11.2 mmol) in CCl₄ (90 mL) was added N-bromo succinimide (2.2 g, 12.3 mmol) and benzoyl peroxide (0.57 g, 2.2 mmol). The mixture was stirred overnight at 80° C. under nitrogen. The mixture was concentrated under reduced pressure and the residue was purified using silica gel chromatography (eluent: 9% EtOAc in PE) to afford 0.9 g (30% yield) of the title compound as a white solid.

### Step 3: Preparation of 2-((5-(4-fluorophenyl)pyrimidin-4-yl)methyl)isoindoline-1,3-dione

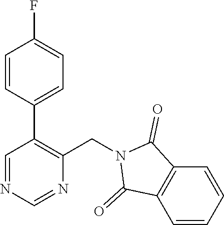 [SMILES: O=C1c2ccccc2C(=O)N1Cc1ncncc1-c1ccc(F)cc1]

Proceeding analogously as described in Example 10, Step 4 but substituting 2-(chloromethyl)-3-(4-fluorophenyl)pyrazine with 4-(bromomethyl)-5-(4-fluorophenyl)pyrimidine provided crude product. The residue was purified using silica gel chromatography (eluent: 17% EtOAc in PE) to afford the title compound as a white solid.

### Step 4: Preparation of (5-(4-fluorophenyl)pyrimidin-4-yl)methanamine

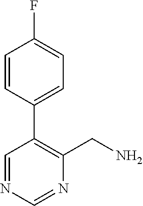 [SMILES: NCc1ncncc1-c1ccc(F)cc1]

The title compound was prepared using General Procedure E employing 2-((5-(4-fluorophenyl)pyrimidin-4-yl)methyl)isoindoline-1,3-dione and hydrazine hydrate (5 eq.). The mixture was stirred overnight at room temperature under nitrogen and then concentrated under reduced pressure. The residue was purified using silica gel chromatography (eluent: 5% MeOH in DCM) to afford the title compound as a white solid.

### Step 5: Preparation of 2-((5-(4-fluorophenyl)pyrimidin-4-yl)methylamino)-4,6-bis(trifluoromethyl)nicotinonitrile

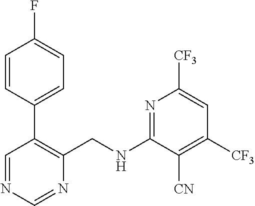 [SMILES: N#Cc1c(C(F)(F)F)cc(C(F)(F)F)nc1NCc1ncncc1-c1ccc(F)cc1]

Proceeding analogously as described in Example 10, Step 6 but substituting (3-(4-fluorophenyl)pyrazin-2-yl)methanamine with (5-(4-fluorophenyl)pyrimidin-4-yl)methanamine afforded crude product. The crude product was purified using silica gel chromatography (eluent: 20% EtOAc in PE) and then by reverse phase Prep-HPLC (column, C₁₈ silica gel; mobile phase, 15-60% ACN in water) to give the title compound as a white solid. ¹H NMR (300 MHz; DMSO-d₆): δ 4.72 (s, 2H), 6.06 (s, 1H), 7.31-7.38 (m, 3H), 7.50-7.54 (m, 2H), 8.66 (s, 1H), 9.10 (s, 1H) ppm. m/z 442 (M+H⁺).

### Example 12

### Synthesis of 2-((4-(4-fluorophenyl)-4H-1,2,4-triazol-3-yl)methylamino)-4,6-bis(trifluoromethyl)nicotinonitrile

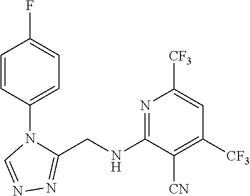 [SMILES: N#Cc1c(C(F)(F)F)cc(C(F)(F)F)nc1NCc1nncn1-c1ccc(F)cc1]

### Step 1: Preparation of 4-fluoro-N-[1-(methylsulfanyl)-2-nitroethenyl]aniline

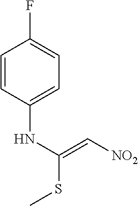

To a solution of 1,1-bis(methylsulfanyl)-2-nitroethene (3.4 g, 20.6 mmol) in EtOH (60 mL) was added 4-fluoroaniline (5.1 g, 45.9 mmol) under nitrogen. The mixture was stirred for 3 h at 80° C. and then filtered and the solid was dried under vacuum to afford 5.1 g of the title compound as an off-white solid.

### Step 2: Preparation of 4-fluoro-N-[1-hydrazinyl-2-nitroethenyl]aniline

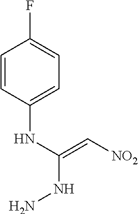 [SMILES: NN/C(=C\[N+](=O)[O-])Nc1ccc(F)cc1]

To a solution of 4-fluoro-N-[1-(methylsulfanyl)-2-nitroethenyl]aniline (5.00 g, 21.9 mmol) in EtOH (60 mL) was added hydrazine (2.70 g, 84.4 mmol) under nitrogen. The mixture was stirred for 4 h at room temperature. The mixture was filtered and the solid was dried under vacuum to afford 4.0 g of the title compound as an off-white solid.

### Step 3: Preparation of 4-(4-fluorophenyl)-3-(nitromethyl)-1,2,4-triazole

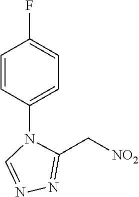 [SMILES: O=[N+]([O-])Cc1nncn1-c1ccc(F)cc1]

To a solution of 4-fluoro-N-[1-hydrazinyl-2-nitroethenyl]aniline (3.95 g, 18.6 mmol) in EtOH(60 mL) was added triethyl orthoformate (5.5 g, 37.3 mmol) under nitrogen. The mixture was stirred for 5 h at 80° C. The mixture was filtered and the solid was dried under vacuum to afford 2.4 g of the title compound as a solid.

### Step 4: Preparation of 1-[4-(4-fluorophenyl)-1,2,4-triazol-3-yl]methanamine

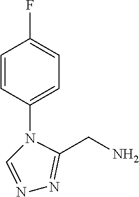 [SMILES: NCc1nncn1-c1ccc(F)cc1]

To a solution of 4-(4-fluorophenyl)-3-(nitromethyl)-1,2,4-triazole (200 mg, 0.9 mmol) in MeOH (20 mL) was added HOAc (4 mL) and 10% Pd/C (10 mg). The mixture was stirred overnight at room temperature under hydrogen. The mixture was filtered and solids washed with MeOH and the filtrate was concentrated under reduced pressure. The residue was purified by reverse flash chromatography (C₁₈ silica gel; 10-50% MeCN in water) to afford 100 mg (50% yield) of the title compound as a white solid.

### Step 5: Preparation of 2-((4-(4-fluorophenyl)-4H-1,2,4-triazol-3-yl)methylamino)-4,6-bis(trifluoromethyl)nicotinonitrile

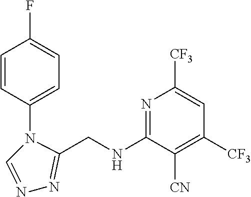 [SMILES: N#Cc1c(C(F)(F)F)cc(C(F)(F)F)nc1NCc1nncn1-c1ccc(F)cc1]

Proceeding analogously as described in Example 6, Step 5, but substituting 1-[1-(4-methoxyphenyl)-1H-imidazol-2-yl]methanamine with 1-[4-(4-fluorophenyl)-1,2,4-triazol-3-yl]methanamine gave crude product. Purification of the crude product by Prep-TLC (20:1, DCM:MeOH) provided the title compound as a white solid. ¹H NMR (300 MHz; DMSO-d₆): δ 8.76-8.71 (m, 2H), 7.55-7.51 (m, 2H), 7.42 (s, 1H), 7.37-7.27 (m, 2H), 4.76 (d, 2H) ppm. m/z 431 (M+H⁺).

### Example 13

### Synthesis of 2-([1-[1-(4-fluorophenyl)imidazol-2-yl]ethyl]amino)-4,6-bis(trifluoromethyl)pyridine-3-carbonitrile

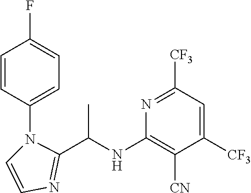 [SMILES: CC(Nc1nc(C(F)(F)F)cc(C(F)(F)F)c1C#N)c1nccn1-c1ccc(F)cc1]

### Step 1: Preparation of 1-[1-(4-fluorophenyl)imidazol-2-yl]ethenone

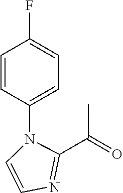

To a solution of ethyl 1-(4-fluorophenyl)-1H-imidazole-2-carboxylate (1.0 g, 4.3 mmol, Example 4, Step 1) in THE (20 mL) was added Et₃N (0.86 g, 8.5 mmol under nitrogen. A solution of MeMgBr in THE (4.3 mL, 4.3 mmol, 1M) was added dropwise at −78° C. and the mixture was stirred for 2 h at −78° C. The mixture was quenched with sat. NH₄Cl and then extracted with EtOAc. The combined organic layers were concentrated to afford 280 mg (32% yield) of the title compound a white solid which was used without further purification.

### Step 2: Preparation of 1-[1-(4-fluorophenyl)imidazol-2-yl]ethanol

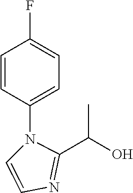

To a solution of 1-[1-(4-fluorophenyl)imidazol-2-yl]ethanone (260 mg, 1.3 mmol) in MeOH (5 mL) was added NaBH₄ (96 mg, 2.5 mmol). The mixture was stirred for 4 h at room temperature and then quenched with sat. NH₄Cl. The mixture was extracted with EtOAc and the combined organic layers were concentrated. The residue was purified by Prep-TLC (30:1, DCM:MeOH) to afford 80 mg (30% yield) of the title compound as a white solid.

### Step 3: Preparation of 2-(1-chloroethyl)-1-(4-fluorophenyl)imidazole

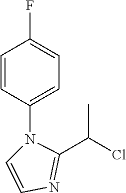

The title compound was prepared using General Procedure B employing 1-[1-(4-fluorophenyl)imidazol-2-yl]ethanol. The mixture was stirred overnight at 50° C. and then concentrated under reduced pressure and the residue was used in the next step without further purification.

### Step 4: Preparation of 2-[1-[1-(4-fluorophenyl)imidazol-2-yl]ethyl]isoindole-1,3-dione

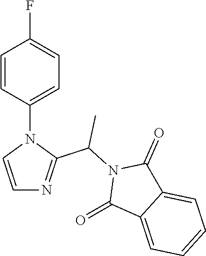 [SMILES: CC(c1nccn1-c1ccc(F)cc1)N1C(=O)c2ccccc2C1=O]

The title compound was prepared using General Procedure D employing 2-(1-chloroethyl)-1-(4-fluorophenyl)imidazole. The mixture was stirred overnight at room temperature and then diluted with water and extracted with EtOAc. The combined organic layers were concentrated under reduced pressure and the was purified using silica gel chromatography (eluent: 33% EtOAc in PE) to afford the title compound as a white solid.

### Step 5: Preparation of 1-[1-(4-fluorophenyl)imidazol-2-yl]ethanamine

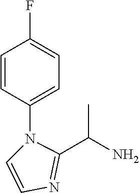

The title compound was prepared using General Procedure E employing 2-[1-[1-(4-fluorophenyl)imidazol-2-yl]ethyl]isoindole-1,3-dione and hydrazine hydrate (5 eq.). The mixture was stirred for 3 h at 50° C. and then filtered. The filtrate was concentrated under reduced pressure to afford 55 mg (86% yield) of the title compound as colorless oil which was used without further purification

### Step 6: Preparation of 2-([1-[1-(4-fluorophenyl)imidazol-2-yl]ethyl]amino)-4,6-bis(trifluoromethyl)pyridine-3-carbonitrile

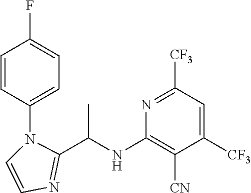 [SMILES: CC(Nc1nc(C(F)(F)F)cc(C(F)(F)F)c1C#N)c1nccn1-c1ccc(F)cc1]

The title compound was prepared using General Procedure E employing 1-[1-(4-fluorophenyl)imidazol-2-yl]ethanamine and 2-chloro-4,6-bis(trifluoromethyl)pyridine-3-carbonitrile (Intermediate A). The mixture was stirred for 4 h at 50° C. and then diluted with EtOAc and washed with water. The organic layer was concentrated and the residue was purified by Prep-TLC with (1:1, EtOAc:PE) to afford the title compound as a white solid. ¹H NMR (400 MHz; DMSO-d₆): δ 8.40 (d, 1H), 7.35-7.42 (m, 2H), 7.32 (d, 2H), 7.14-7.17 (m, 2H), 7.01 (s, 1H), 5.43-5.47 (m, 1H), 1.52 (d, 3H) ppm. m/z 444 (M+H⁺).

### Example 14

### Synthesis of 2-(((4-(4-fluorophenyl)-1-methyl-1H-pyrazol-3-yl)methyl)amino)-4,6-bis(trifluoromethyl)nicotinonitrile

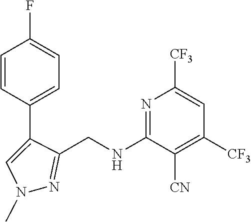 [SMILES: Cn1cc(-c2ccc(F)cc2)c(CNc2nc(C(F)(F)F)cc(C(F)(F)F)c2C#N)n1]

### Step 1: Preparation of methyl 4-(4-fluorophenyl)-1-methylpyrazole-3-carboxylate

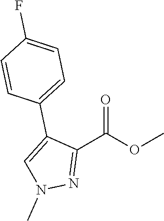 [SMILES: COC(=O)c1nn(C)cc1-c1ccc(F)cc1]

To a solution of methyl 4-bromo-1-methylpyrazole-3-carboxylate (3.0 g, 13.7 mmol) in 1,4-dioxane (50 mL) was added 4-fluorophenylboronic acid (3.8 g, 27.4 mmol), K₂CO₃ (5.7 g, 41.1 mmol) and Pd(PPh₃)₄ (2.4 g, 2.1 mmol). The mixture was stirred for 2 h at 100° C., diluted with water and extracted with EtOAc. The combined organic layers were concentrated under reduced pressure and the residue was purified using silica gel chromatography (eluent: 20% EtOAc in PE) to give the title compound (2.2 g, 68% yield) as a light yellow solid.

### Step 2: Preparation of [4-(4-fluorophenyl)-1-methylpyrazol-3-yl]methanol

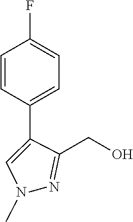

The title compound was prepared using General Procedure A employing methyl 4-(4-fluorophenyl)-1-methylpyrazole-3-carboxylate and stirring the mixture at −78° C. A saturated solution of NH₄Cl in water was added and the mixture was warmed to rt. The mixture was extracted with EtOAc and the combined organic layers were concentrated under reduced pressure. The residue was purified using silica gel chromatography (eluent: 10% MeOH in DCM) to give the title compound (1.3 g, 69% yield) as a light yellow solid.

### Step 3: Preparation of 3-(chloromethyl)-4-(4-fluorophenyl)-1-methylpyrazole

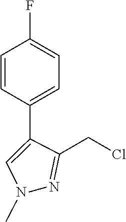

The title compound was prepared using General Procedure B employing [4-(4-fluorophenyl)-1-methylpyrazol-3-yl]methanol.

### Step 4: Preparation of 1-[4-(4-fluorophenyl)-1-methylpyrazol-3-yl]methanamine

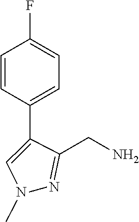

The title compound was prepared using General Procedure C employing 3-(chloro-methyl)-4-(4-fluorophenyl)-1-methylpyrazole. The mixture was stirred at rt for 1 h and then concentrated under reduced pressure. The residue was purified by Prep-TLC (10:1, DCM:MeOH) to afford the title compound (220 mg, 24% yield) as a light yellow solid.

### Step 5: Preparation of 2-(((4-(4-fluorophenyl)-1-methyl-1H-pyrazol-3-yl)methyl)amino)-4,6-bis(trifluoromethyl)nicotinonitrile

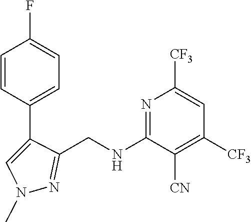 [SMILES: Cn1cc(-c2ccc(F)cc2)c(CNc2nc(C(F)(F)F)cc(C(F)(F)F)c2C#N)n1]

The title compound was prepared using General Procedure F employing 2-chloro-4,6-bis(trifluoromethyl)pyridine-3-carbonitrile (Intermediate A) and 1-[4-(4-fluorophenyl)-1-methylpyrazol-3-yl]methanamine. The mixture was diluted with EtOAc and washed with 1M LiCl. The organic layer was concentrated under reduced pressure and the residue was purified by Prep-TLC (20:1, DCM:MeOH) to afford the title compound (208 mg, 88% yield) as a light yellow solid. ¹H NMR (400 MHz; CDCl₃): δ 8.44-8.41 (t, 1H), 7.86 (s, 1H), 7.42-7.37 (m, 3H), 7.17-7.11 (m, 2H), 4.72-4.73 (d, 2H), 3.82 (s, 3H) ppm. m/z 444 (M+H⁺).

### Example 15

### Synthesis of 2-(((4-(4-fluorophenyl)-1H-pyrazol-3-yl)methyl)amino)-4,6-bis(trifluoromethyl)-nicotinonitrile

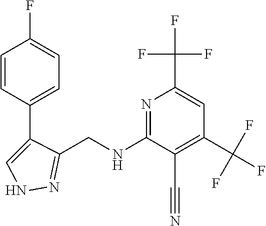 [SMILES: N#Cc1c(C(F)(F)F)cc(C(F)(F)F)nc1NCc1n[nH]cc1-c1ccc(F)cc1]

### Step 1: Preparation of methyl 4-(4-fluorophenyl)-1H-pyrazole-3-carboxylate

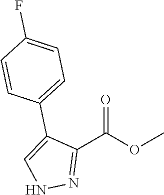 [SMILES: COC(=O)c1n[nH]cc1-c1ccc(F)cc1]

To a solution of methyl 4-bromo-1H-pyrazole-3-carboxylate (5.0 g, 24.3 mmol) in 1,4-dioxane (100 mL) was added 4-fluorophenylboronic acid (6.8 g, 48.6 mmol), K₂CO₃ (10.1 g, 72.9 mmol) and Pd(PPh₃)₄ (4.2 g, 3.7 mmol). The mixture was stirred at 100° C. for 5 h. The mixture was cooled to rt and diluted with water. The mixture was extracted with EtOAc (2×). The combined organic layers were concentrated, and the residue was purified using silica gel chromatography (eluent: 2-50% EtOAc in petroleum ether) to give methyl 4-(4-fluorophenyl)-1H-pyrazole-3-carboxylate (2.0 g, 37% yield) as a light yellow solid.

### Step 2: Preparation of (4-(4-fluorophenyl)-1H-pyrazol-3-yl)methanol

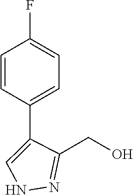 [SMILES: OCc1n[nH]cc1-c1ccc(F)cc1]

The title compound was prepared using General Procedure A employing methyl 4-(4-fluorophenyl)-1H-pyrazole-3-carboxylate. The mixture was diluted with a solution of saturated NH₄Cl in water slowly. The mixture was filtered through celite and the filtrate was extracted with EtOAc. The combined organic layers were concentrated, and the residue was triturated with DCM to give the title compound (1.1 g, 63% yield) as a white solid.

### Step 3: Preparation of 3-(chloromethyl)-4-(4-fluorophenyl)-1H-pyrazole

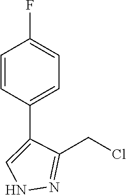 [SMILES: Fc1ccc(-c2c[nH]nc2CCl)cc1]

The title compound was prepared using General Procedure B employing [4-(4-fluorophenyl)-1H-pyrazol-3-yl]methanol. The mixture was diluted with MTBE and stirred at rt for 15 minutes and then filtered to give the title compound as a white solid.

### Step 4: Preparation of 2-((4-(4-fluorophenyl)-1H-pyrazol-3-yl)methyl)isoindoline-1,3-dione

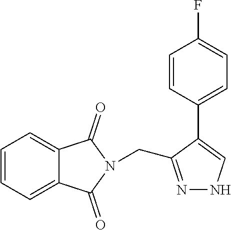 [SMILES: O=C1c2ccccc2C(=O)N1Cc1n[nH]cc1-c1ccc(F)cc1]

The title compound was prepared using General Procedure D employing 3-(chloromethyl)-4-(4-fluorophenyl)-1H-pyrazole. The mixture was diluted with water and filtered and the solid was purified by prep-TLC (20:1, DCM:MeOH) to afford the title compound as a white solid.

### Step 5: Preparation of 1-[4-(4-fluorophenyl)-1H-pyrazol-3-yl]methanamine

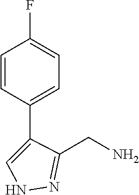 [SMILES: NCc1n[nH]cc1-c1ccc(F)cc1]

The title compound was prepared using General Procedure E employing 2-[[4-(4-fluorophenyl)-1H-pyrazol-3-yl]methyl]isoindole-1,3-dione. The mixture was cooled to rt and diluted with EtOH. The mixture was filtered, and the filtrate was concentrated under reduced pressure to give the title compound as a white solid.

### Step 6: Preparation of 2-(((4-(4-fluorophenyl)-1H-pyrazol-3-yl)methyl)amino)-4,6-bis(trifluoromethyl)nicotinonitrile

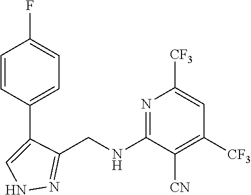 [SMILES: N#Cc1c(C(F)(F)F)cc(C(F)(F)F)nc1NCc1n[nH]cc1-c1ccc(F)cc1]

The title compound was prepared using General Procedure F employing 2-chloro-4,6-bis(trifluoromethyl)pyridine-3-carbonitrile (Intermediate A) and 1-[4-(4-fluorophenyl)-1H-pyrazol-3-yl]methanamine. The mixture was diluted with EtOAc and washed with 1M LiCl (2×). The organic layer was concentrated under reduced pressure. The residue was purified by Prep-TLC (20:1, DCM:MeOH) to afford the title compound as a light yellow solid. ¹H NMR (400 MHz; DMSO-d₆): δ 12.78 (d, 1H), 8.62-8.38 (m, 1H), 7.80 (d, 1H), 7.49-7.38 (m, 3H), 7.22-7.13 (m, 2H), 4.86-4.76 (m, 2H) ppm. m/z 430 (M+H⁺).

### Example 16

### Synthesis of 2-(((3-(4-fluorophenyl)pyridin-2-yl)methyl)amino)-4,6-bis(trifluoromethyl)-nicotinonitrile

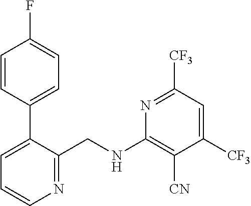 [SMILES: N#Cc1c(C(F)(F)F)cc(C(F)(F)F)nc1NCc1ncccc1-c1ccc(F)cc1]

### Step 1: Preparation of 3-(4-fluorophenyl)picolinonitrile

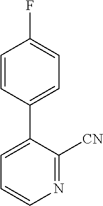 [SMILES: N#Cc1ncccc1-c1ccc(F)cc1]

To a solution of 3-bromopyridine-2-carbonitrile (2.0 g, 10.9 mmol) and 4-fluorophenyl-boronic acid (4.6 g, 32.8 mmol) in 1,4-dioxane (20 mL) was added K₂CO₃ (3.0 g, 21.9 mmol) and [1,1-bis(di-tert-butylphosphino)ferrocene]dichloropalladium(II) (712 mg, 1.1 mmol). The mixture was stirred overnight at 80° C. under nitrogen. A solution of saturated NH₄Cl in water was added and the mixture was extracted with EtOAc. The combined organic layers were concentrated under reduced pressure and the residue was purified using silica gel chromatography (eluent: 100% DCM) to afford the title compound (1.7 g, 83% yield) as a white solid.

### Step 2: Preparation of 1-[3-(4-fluorophenyl)pyridin-2-yl]methanamine hydrochloride

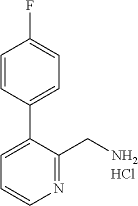 [SMILES: NCc1ncccc1-c1ccc(F)cc1 ; Cl]

To a solution of 3-(4-fluorophenyl)pyridine-2-carbonitrile (1.7 g, 9.1 mmol) in MeOH (80 mL) and HCl (12 M, 1.6 mL) was added 10% Pd/C (170 mg) and the solution was stirred at rt overnight over under an atmosphere of hydrogen (1 atm). The mixture was filtered, and the filtrate was concentrated under reduced pressure to afford the title compound (2.4 g, 98% yield) as an off-white solid.

### Step 3: Preparation of 2-(((3-(4-fluorophenyl)pyridin-2-yl)methyl)amino)-4,6-bis(trifluoromethyl)nicotinonitrile

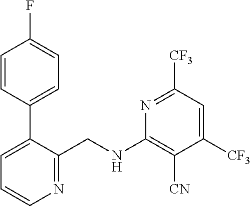 [SMILES: N#Cc1c(C(F)(F)F)cc(C(F)(F)F)nc1NCc1ncccc1-c1ccc(F)cc1]

The title compound was prepared using General Procedure F employing 2-chloro-4,6-bis(trifluoromethyl)pyridine-3-carbonitrile (Intermediate A) and 1-[3-(4-fluorophenyl)pyridin-2-yl]methanamine hydrochloride using 3 eq. of DIEA. The mixture was diluted with EtOAc and washed with 1M LiCl. The organic layers were concentrated under reduced pressure and the residue was purified by Prep-TLC (4:1, PE:EtOAc) to afford the title compound (226 mg, 52% yield) as an off-white solid. ¹H NMR (300 MHz; DMSO-d₆): δ 8.57-8.52 (m, 2H), 7.69-7.66 (m, 1H), 7.50-7.25 (m, 6H), 4.70 (d, 2H) ppm. m/z 441 (M+H⁺).

### Example 17

### Synthesis of 2-([[3-(2-chloro-4-fluorophenyl) pyrazin-2-yl] methyl] amino)-4,6-bis(trifluoromethyl)pyridine-3-carbonitrile

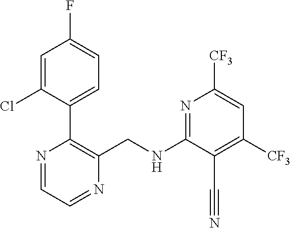 [SMILES: N#Cc1c(C(F)(F)F)cc(C(F)(F)F)nc1NCc1nccnc1-c1ccc(F)cc1Cl]

### Step 1: Preparation of methyl 3-(2-chloro-4-fluorophenyl) pyrazine-2-carboxylate

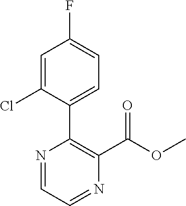 [SMILES: COC(=O)c1nccnc1-c1ccc(F)cc1Cl]

To a solution of methyl 3-bromopyrazine-2-carboxylate (2.0 g, 9.2 mmol) in 1,4-dioxane (20 mL) was added 2-chloro-4-fluorophenylboronic acid (3.2 g, 18.4 mmol), K₂CO₃ (2.6 g, 18.4 mmol), and Pd(dppf)Cl₂ (1.0 g, 1.34 mmol). The mixture was stirred for 8 hr at 80° C. and then cooled to room temperature and diluted with water. The mixture was extracted with DCM and the combined organic layers were concentrated under reduced pressure. The residue was purified using silica gel chromatography (eluent: 9% EtOAc in petroleum ether) to afford the title compound (1.3 g, 50% yield) as an off-white solid.

### Step 2: Preparation of (3-(2-chloro-4-fluorophenyl)pyrazin-2-yl)methanol

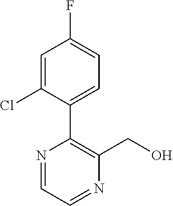 [SMILES: OCc1nccnc1-c1ccc(F)cc1Cl]

To a solution of methyl 3-(2-chloro-4-fluorophenyl)pyrazine-2-carboxylate (1.3 g, 4.9 mmol) in DCM (20 mL) at −78° C. was added DIBAl-H (1.2 g, 8.6 mmol) and the mixture was stirred for 3 h at −78° C. The mixture was warmed to rt and a solution of saturated NH₄Cl in water was added. The mixture was extracted with DCM and the combined organic layers were concentrated under vacuum. The residue was purified using silica gel chromatography (eluent: 9% EtOAc in hexanes) to afford the title compound (370 mg, 32% yield) as a yellow oil.

### Step 3: Preparation of 2-(2-chloro-4-fluorophenyl)-3-(chloromethyl) pyrazine

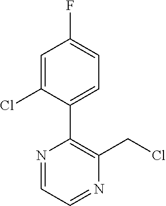 [SMILES: Fc1ccc(-c2nccnc2CCl)c(Cl)c1]

The title compound was prepared using General Procedure B employing [3-(2-chloro-4-fluorophenyl) pyrazin-2-yl] methanol.

### Step 4: Preparation of 2-[[3-(2-chloro-4-fluorophenyl) pyrazin-2-yl] methyl] isoindole-1, 3-dione

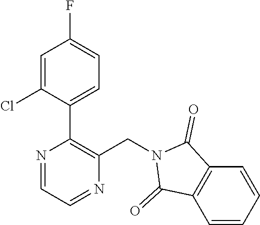 [SMILES: O=C1c2ccccc2C(=O)N1Cc1nccnc1-c1ccc(F)cc1Cl]

The title compound was prepared using General Procedure D employing 2-(2-chloro-4-fluorophenyl)-3-(chloromethyl)pyrazine. The mixture was diluted with water and extracted with DCM. The combined organic layers were concentrated under reduced pressure and the residue was triturated with petroleum ether to afford the title compound (600 mg) as a brown solid.

### Step 5: Preparation of 1-[3-(2-chloro-4-fluorophenyl) pyrazin-2-yl]methanamine

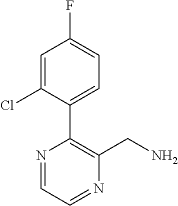 [SMILES: NCc1nccnc1-c1ccc(F)cc1Cl]

The title compound was prepared using General Procedure E employing 2-[[3-(2-chloro-4-fluorophenyl) pyrazin-2-yl]methyl]isoindole-1,3-dione. The mixture was filtered, and the filtrate was concentrated under vacuum to afford the title compound as a brown oil.

### Step 6: Preparation of 2-([[3-(2-chloro-4-fluorophenyl) pyrazin-2-yl] methyl] amino)-4,6-bis(trifluoromethyl)pyridine-3-carbonitrile

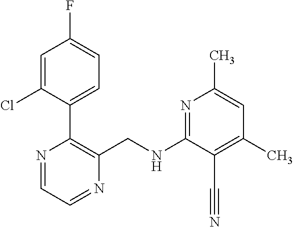 [SMILES: Cc1cc(C)c(C#N)c(NCc2nccnc2-c2ccc(F)cc2Cl)n1]

The title compound was prepared using General Procedure F employing 2-chloro-4,6-bis(trifluoromethyl)pyridine-3-carbonitrile (Intermediate A) and 1-[3-(2-chloro-4-fluorophenyl) pyrazin-2-yl]methanamine. The mixture was diluted with water and extracted with DCM. The combined organic layers were concentrated under reduced pressure and the residue was purified by Prep-TLC (1:1, PE:EtOAc) to afford the title compound (28 mg, 12% yield) as a light orange solid. ¹H NMR (400 MHz; DMSO-d₆): δ 8.67 (s, 2H), 8.61 (t, 1H), 7.54-7.46 (m, 2H), 7.37 (s, 1H), 7.34-7.30 (m, 1H), 4.70 (s, 2H) ppm. m/z 476 (M+H⁺).

### Example 18

### Synthesis of 2-([[5-(pyridin-4-yl)pyrimidin-4-yl]methyl]amino)-4,6-bis(trifluoromethyl)pyridine-3-carbonitrile

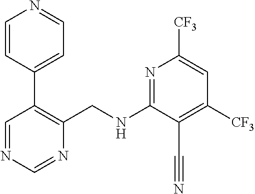 [SMILES: N#Cc1c(C(F)(F)F)cc(C(F)(F)F)nc1NCc1ncncc1-c1ccncc1]

### Step 1: Preparation of 5-bromo-4-(bromomethyl)pyrimidine as yellow oil

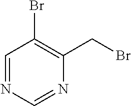

To a solution of 5-bromo-4-methylpyrimidine in AcOH (2 mL) was added Br₂ (554 mg, 3.5 mmol) and the mixture was stirred for 40 min at 80° C. The mixture was cooled to rt and a saturated solution of NaHCO₃ in water was added. The mixture was extracted with EtOAc and then concentrated under reduced pressure to afford the title compound which was used in the next step without further purification.

### Step 2: Preparation of 2-[(5-bromopyrimidin-4-yl)methyl]isoindole-1,3-dione

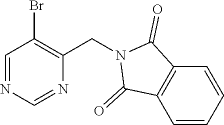 [SMILES: O=C1c2ccccc2C(=O)N1Cc1ncncc1Br]

The title compound was prepared using General Procedure D employing 5-bromo-4-(bromomethyl)pyrimidine. The mixture was diluted with water and the mixture was filtered. The solids were collected to afford the title compound (780 mg, 76% yield) as a light pink solid.

### Step 3: Preparation of 2-[[5-(pyridin-4-yl) pyrimidin-4-yl] methyl] isoindole-1,3-dione

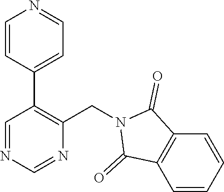 [SMILES: O=C1c2ccccc2C(=O)N1Cc1ncncc1-c1ccncc1]

To a solution of 2-[(5-bromopyrimidin-4-yl)methyl]isoindole-1,3-dione (780.00 mg, 2.452 mmol) in 1,4-dioxane (10 mL) and water (2.5 mL) was added pyridin-4-ylboronic acid (603 mg, 4.9 mmol), Cs₂CO₃ (1.6 g, 4.9 mmol), and Pd(dppf)Cl₂ (269 mg, 0.37 mmol). The mixture was stirred overnight at 100° C. and then diluted with water and extracted with EtOAc. The combined organic layers were concentrated under reduced pressure. The residue was purified using silica gel chromatography (eluent: 33% EtOAc in hexanes) to afford the title compound (650 mg, 80% yield) as an off-white solid.

### Step 4: Preparation of 1-[5-(pyridin-4-yl) pyrimidin-4-yl] methanamine

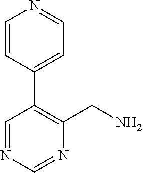 [SMILES: NCc1ncncc1-c1ccncc1]

The title compound was prepared using General Procedure E employing 2-[[5-(pyridin-4-yl)pyrimidin-4-yl]methyl]isoindole-1,3-dione. The mixture was cooled to rt and filtered. The filtrate was concentrated under vacuum to afford the title compound as a brown oil.

### Step 5: Preparation of 2-([[5-(pyridin-4-yl)pyrimidin-4-yl]methyl]amino)-4,6-bis(trifluoromethyl)pyridine-3-carbonitrile

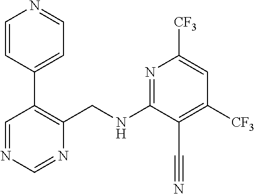 [SMILES: N#Cc1c(C(F)(F)F)cc(C(F)(F)F)nc1NCc1ncncc1-c1ccncc1]

The title compound was prepared using General Procedure F employing 2-chloro-4,6-bis(trifluoromethyl)pyridine-3-carbonitrile (Intermediate A) and 1-[5-(pyridin-4-yl) pyrimidin-4-yl]methanamine. The mixture was stirred overnight at room temperature and then diluted with EtOAc. The mixture was washed with 1M LiOH and the organic layer was concentrated under reduced pressure. The residue was purified by Prep-TLC (10:1, DCM:MeOH) to afford the title compound (111 mg, 16% yield) as a light yellow solid. ¹H NMR (300 MHz; DMSO-d₆): δ 9.18 (s, 1H), 8.81-8.68 (m, 4H), 7.54-7.52 (m, 2H), 7.37 (s, 1H), 4.75 (s, 2H) ppm. m/z 425 (M+H⁺).

### Example 19

### Synthesis of 2-[[3-(4-fluorophenyl)pyridin-2-yl]methoxy]-4,6-bis(trifluoromethyl)pyridine-3-carbonitrile

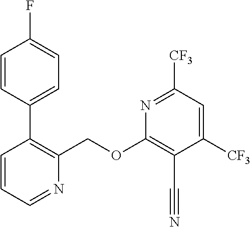 [SMILES: N#Cc1c(C(F)(F)F)cc(C(F)(F)F)nc1OCc1ncccc1-c1ccc(F)cc1]

### Step 1: Preparation of methyl 3-(4-fluorophenyl)pyridine-2-carboxylate

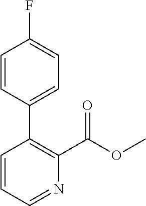 [SMILES: COC(=O)c1ncccc1-c1ccc(F)cc1]

To a solution of methyl 3-bromopyridine-2-carboxylate (1.0 g, 4.6 mmol) and 4-fluorophenylboronic acid (972 mg, 6.9 mmol) in 1,4-dioxane (8 mL) and water (2 mL) was added Pd(dppf)Cl₂ (334 mg, 0.46 mmol) and K₂CO₃ (1279 mg, 9.26 mmol). The mixture was stirred at 100° C. overnight and then cooled to room temperature and diluted with EtOAc. The mixture was washed with water and the organic layer was concentrated under reduced pressure. The residue was purified by silica gel column chromatography (eluent: 100% DCM) to afford the title compound as a yellow solid.

### Step 2: Preparation of [3-(2-chlorophenyl)pyridin-2-yl]methanol

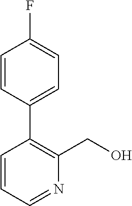 [SMILES: OCc1ncccc1-c1ccc(F)cc1]

To a solution of methyl 3-(2-chlorophenyl)pyridine-2-carboxylate (1.4 g, 5.7 mmol) in MeOH (14 mL) was added NaBH₄ (428 mg, 11.3 mmol) and CaCl₂ (1903 mg, 11.3 mmol). The mixture was stirred at 60° C. overnight, then cooled to room temperature and diluted with saturated NH₄Cl (aq.). The aqueous layer was extracted with EtOAc and the combined organic layers were concentrated under reduced pressure. The residue was purified by silica gel column chromatography, eluted with 9% EtOAc in PE to afford the title compound (500 mg, 40% yield) as a yellow solid.

### Step 3: Preparation of 2-[[3-(4-fluorophenyl)pyridin-2-yl]methoxy]-4,6-bis(trifluoromethyl)-pyridine-3-carbonitrile

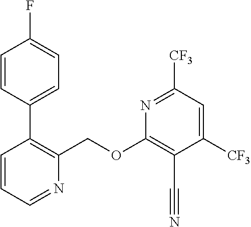 [SMILES: N#Cc1c(C(F)(F)F)cc(C(F)(F)F)nc1OCc1ncccc1-c1ccc(F)cc1]

To a solution of [3-(4-fluorophenyl)pyridin-2-yl]methanol (100 mg, 0.49 mmol) in MeCN (1 mL) was added 2-chloro-4,6-bis(trifluoromethyl)pyridine-3-carbonitrile (Intermediate A, 135 mg, 0.49 mmol) and K₂CO₃ (136 mg, 0.98 mmol). The mixture was stirred at 80° C. overnight, cooled to room temperature and diluted with water. The aqueous layer was extracted with EtOAc and the combined organic layers were concentrated under reduced pressure. The residue was purified by Prep-TLC (80:1, DCM:MeOH) to afford the title compound (43 mg, 20% yield) as a white solid. ¹H NMR (300 MHz; DMSO-d₆): δ 8.60-8.58 (m, 1H), 8.03 (s, 1H), 7.79-7.76 (m, 1H), 7.51-7.44 (m, 3H), 7.27-7.21 (m, 2H), 5.68 (s, 2H) ppm. m/z 442 (M+H⁺).

### Example 20

### Synthesis of 2-([[3-(4-fluorophenyl)-6-methylpyridin-2-yl]methyl]amino)-4,6-bis(trifluoromethyl)pyridine-3-carbonitrile

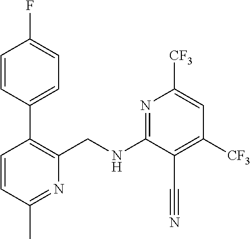 [SMILES: Cc1ccc(-c2ccc(F)cc2)c(CNc2nc(C(F)(F)F)cc(C(F)(F)F)c2C#N)n1]

### Step 1: Preparation of 3-(4-fluorophenyl)-6-methylpyridine-2-carbonitrile

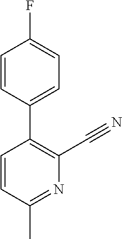 [SMILES: Cc1ccc(-c2ccc(F)cc2)c(C#N)n1]

To a solution of 3-bromo-6-methylpyridine-2-carbonitrile (1.0 g, 5.1 mmol) in 1,4-dioxane (16 mL) and water (4 mL) was added 4-fluorophenylboronic acid (1.4 g, 10.1 mmol), K₂CO₃ (1.4 g, 10.2 mmol) and Pd(dppf)Cl₂ (0.37 g, 0.51 mmol) at room temperature. The mixture was stirred at 100° C. for 3 h under nitrogen atmosphere, then diluted with EtOAc and washed with water. The organic layer was concentrated under reduced pressure and the residue was purified by silica gel column chromatography (eluent: with 9% EtOAc in PE) to afford the title compound (1 g, 93% yield) as a white solid.

### Step 2: Preparation of 1-[3-(4-fluorophenyl)-6-methylpyridin-2-yl]methanamine

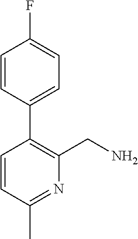 [SMILES: Cc1ccc(-c2ccc(F)cc2)c(CN)n1]

To a solution of 3-(4-fluorophenyl)-6-methylpyridine-2-carbonitrile (300 mg, 1.4 mmol) in MeOH (80 mL) and HOAc (20 mL) was added 10% Pd/C (60 mg) at room temperature and the mixture was stirred for 2 h at room temperature under an atmosphere of hydrogen (1 atm). The mixture was filtered, the filter cake was washed with MeOH and the filtrate was concentrated under reduced pressure to give the title compound (300 mg) as a colorless liquid

### Step 3: Preparation of 2-([[3-(4-fluorophenyl)-6-methylpyridin-2-yl]methyl]amino)-4,6-bis(trifluoromethyl)pyridine-3-carbonitrile

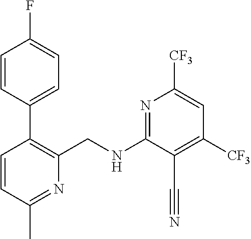 [SMILES: Cc1ccc(-c2ccc(F)cc2)c(CNc2nc(C(F)(F)F)cc(C(F)(F)F)c2C#N)n1]

The title compound was prepared using General Procedure F employing 1-[3-(4-fluorophenyl)-6-methylpyridin-2-yl]methanamine and 2-chloro-4,6-bis(trifluoromethyl)pyridine-3-carbonitrile (Intermediate A). The mixture was cooled to room temperature and diluted with EtOAc and the resulting mixture was washed with water. The mixture was concentrated under reduced pressure and the residue was purified by Prep-TLC (5:1, PE:EtOAc) to afford the title compound (24 mg, 11% yield) as a white solid. ¹H NMR (300 MHz; DMSO-d₆): δ 8.48 (t, 1H), 7.65 (d, 1H), 7.58-7.41 (m, 3H), 7.39-7.22 (m, 3H), 4.67 (d, 2H), 2.49 (s, 3H) ppm. m/z 455 (M+H⁺).

### Example 21 Synthesis of 2-(((3-(pyridin-2-yl)pyrazin-2-yl)methyl)amino)-4,6-his(trifluoromethyl)-nicotinonitrile

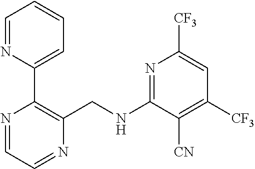 [SMILES: N#Cc1c(C(F)(F)F)cc(C(F)(F)F)nc1NCc1nccnc1-c1ccccn1]

### Step 1: Preparation of 3-(pyridin-2-yl)pyrazine-2-carbonitrile

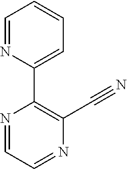 [SMILES: N#Cc1nccnc1-c1ccccn1]

To a solution of 3-chloropyrazine-2-carbonitrile (1.0 g, 7.2 mmol) in toluene (10 mL) was added 2-(tributylstannyl)pyridine (2.9 g, 7.9 mmol), and Pd(PPh₃)₄ (828 mg, 0.72 mmol) under nitrogen. The mixture was stirred at 110° C. for 3 hours and then concentrated under reduced pressure. The residue was purified by silica gel column chromatography (eluent: 13% EtOAc in PE) to afford the title compound (540 mg, 40% yield) as an off-white solid.

### Step 2: Preparation of 1-[3-(pyridin-2-yl)pyrazin-2-yl]methanamine

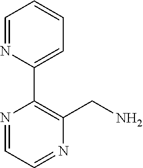 [SMILES: NCc1nccnc1-c1ccccn1]

To a solution of 3-(pyridin-2-yl)pyrazine-2-carbonitrile (500 mg, 2.7 mmol) in MeOH (20 mL) and HOAc (5 mL) was added 10% Pd/C (100 mg, 0.94 mmol). The mixture was stirred for 1 hour at room temperature under an atmosphere of hydrogen. The mixture was filtered and the filtrate was concentrated under vacuum to afford the title compound (390 mg, 56% yield) as a black oil.

### Step 3: Preparation of 2-(((3-(pyridin-2-yl)pyrazin-2-yl)methyl)amino)-4,6-bis(trifluoromethyl)-nicotinonitrile

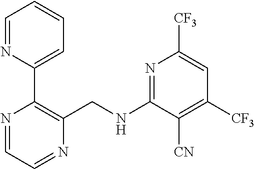 [SMILES: N#Cc1c(C(F)(F)F)cc(C(F)(F)F)nc1NCc1nccnc1-c1ccccn1]

The title compound was prepared using General Procedure F employing 1-[3-(pyridin-2-yl)pyrazin-2-yl]methanamine and 2-chloro-4,6-bis(trifluoromethyl)pyridine-3-carbonitrile (Intermediate A). The mixture was stirred for 3 h at room temperature and then diluted with water and extracted with EtOAc. The combined organic layers were concentrated under reduced pressure and the residue was purified by Prep-TLC (20:1, DCM:MeOH) to afford the title compound as an off-white solid. ¹H NMR (300 MHz; DMSO-d₆): δ 8.78-8.73 (m, 2H), 8.67-8.64 (m, 2H), 8.13-8.02 (m, 2H), 7.57-7.53 (m, 1H), 7.36 (s, 1H), 5.14-5.12 (m, 2H) ppm. m/z 425 (M+H⁺).

### Example 22

### Synthesis of 2-[[2,3′-bipyridine]-2′-carboximidoyl]-4,6-bis(trifluoromethyl)-1,2-dihydropyridine-3-carbonitrile

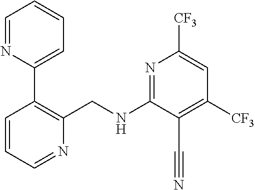 [SMILES: N#Cc1c(C(F)(F)F)cc(C(F)(F)F)nc1NCc1ncccc1-c1ccccn1]

### Step 1: Preparation of [2,3′-bipyridine]-2′-carbonitrile

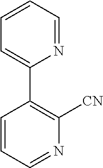 [SMILES: N#Cc1ncccc1-c1ccccn1]

To a solution of 3-bromopyridine-2-carbonitrile (1.0 g, 5.5 mmol) in toluene (10 mL) was added 3-(tributylstannyl)pyridine (2.2 g, 6.0 mmol) in portions under an atmosphere of nitrogen. Pd(PPh₃)₄ (1.3 g, 1.1 mmol) was added and the mixture was stirred for 4 h at room temperature. The mixture was diluted with water and extracted with EtOAc and the combined organic layers were concentrated under reduced pressure. The residue was purified by Prep-TLC (5:1, PE:EtOAc) to afford the title compound as a light yellow solid (400 mg, 89% yield).

### Step 2: Preparation of 1-[[2,3′-bipyridin]-2′-yl]methanamine

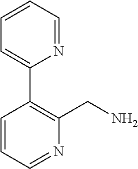 [SMILES: NCc1ncccc1-c1ccccn1]

To a solution of [2,3′-bipyridine]-2′-carbonitrile (200 mg, 1.1 mmol) in MeOH (20 mL) and AcOH (80 mL) was added 10% Pd/C (400 mg). The mixture was stirred for 2 h at room temperature under an atmosphere of hydrogen (1 atm), then filtered and the filtrate was concentrated under reduce pressure. The residue was purified by Prep-TLC (20:1, DCM:MeOH) to afford the title compound (120 mg, 88% yield) as a light yellow solid.

### Step 3: Preparation of 2-[[2,3′-bipyridine]-2′-carboximidoyl]-4,6-bis(trifluoromethyl)-1,2-dihydropyridine-3-carbonitrile

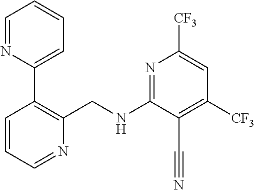 [SMILES: N#Cc1c(C(F)(F)F)cc(C(F)(F)F)nc1NCc1ncccc1-c1ccccn1]

The title compound was prepared using General Procedure F employing 1-[[2,3′-bipyridin]-2′-yl]methanamine and 2-chloro-4,6-bis(trifluoromethyl)pyridine-3-carbonitrile (Intermediate A). The mixture was stirred for 2 h at 50° C. and then washed with water. The aqueous layers were extracted with EtOAc and the combined organic layers were concentrated under reduced pressure. The residue was purified by Prep-TLC (1:1, PE:EtOAc) to afford the title compound (70 mg, 99% yield) as a white solid. ¹H NMR (300 MHz; DMSO-d₆): δ 8.70-8.64 (m, 1H), 8.63-8.59 (m, 2H), 8.00-7.93 (m, 2H), 7.71-7.68 (m, 1H), 7.48-7.43 (m, 2H), 7.36 (s, 1H), 4.89 (d, 2H) ppm. m/z 424 (M+H⁺).

### Example 23

### Synthesis of 2-([[3-(4-fluorophenyl)-6-oxo-1H-pyridin-2-yl]methyl]amino)-4,6-bis(trifluoromethyl)pyridine-3-carbonitrile

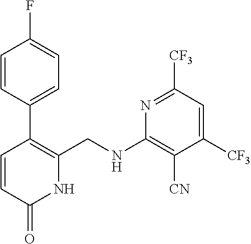 [SMILES: N#Cc1c(C(F)(F)F)cc(C(F)(F)F)nc1NCc1[nH]c(=O)ccc1-c1ccc(F)cc1]

### Step 1: Preparation of 6-chloro-3-(4-fluorophenyl)pyridine-2-carbonitrile

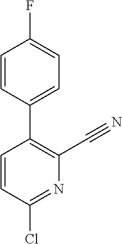 [SMILES: N#Cc1nc(Cl)ccc1-c1ccc(F)cc1]

To a solution of 3-bromo-6-chloropyridine-2-carbonitrile (2.0 g, 9.2 mmol) and 4-fluorophenylboronic acid (6.43 mg, 0.046 mmol) in 1,4-dioxane (40 mL) and water (10 mL) was added K₂CO₃ (2.54 g, 18.4 mmol) and Pd(dppf)Cl₂ (0.67 g, 0.92 mmol) under a nitrogen atmosphere. The mixture was stirred for 6 h at 80° C. under a nitrogen atmosphere and then cooled to room temperature and diluted with EtOAc. The organic layer was washed with water and concentrated under reduced pressure. The residue was purified by silica gel column chromatography (eluent: 9% EtOAc in PE) to afford the title compound (2.1 g, 90% yield) as a white solid.

### Step 2: Preparation of 1-[6-chloro-3-(4-fluorophenyl)pyridin-2-yl]methanamine

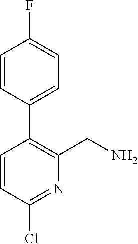 [SMILES: NCc1nc(Cl)ccc1-c1ccc(F)cc1]

To a solution of 6-chloro-3-(4-fluorophenyl)pyridine-2-carbonitrile (2.0 g, 8.6 mmol) in THF (10 mL) was added BH₃ (2 M in THF, 8.6 mL, 17.2 mmol) at room temperature under nitrogen atmosphere. The mixture was stirred for 4 h at room temperature and then concentrated under reduced pressure. The residue was purified by Prep-TLC (10:1, DCM:MeOH) to give the title compound (368 mg, 14% yield) as a yellow solid.

### Step 3: Preparation of 2-([[6-chloro-3-(4-fluorophenyl)pyridin-2-yl]methyl]amino)-4,6-bis(trifluoromethyl)pyridine-3-carbonitrile

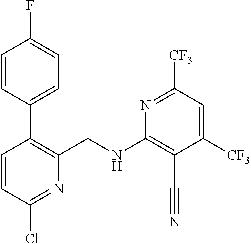 [SMILES: N#Cc1c(C(F)(F)F)cc(C(F)(F)F)nc1NCc1nc(Cl)ccc1-c1ccc(F)cc1]

The title compound was prepared using General Procedure F employing 1-[6-chloro-3-(4-fluorophenyl)pyridin-2-yl]methanamine and 2-chloro-4,6-bis(trifluoromethyl)pyridine-3-carbonitrile (Intermediate A). The mixture was stirred overnight at room temperature and then diluted with EtOAc and washed with water. The organic layer was concentrated under reduced pressure and the residue was purified by Prep-TLC (7:1, PE:EtOAc) to afford the title compound (90 mg, 12% yield) as a white solid.

### Step 4: Preparation of 2-([[3-(4-fluorophenyl)-6-oxo-1H-pyridin-2-yl]methyl]amino)-4,6-bis(trifluoromethyl)pyridine-3-carbonitrile

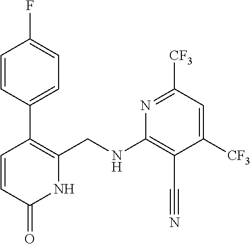 [SMILES: N#Cc1c(C(F)(F)F)cc(C(F)(F)F)nc1NCc1[nH]c(=O)ccc1-c1ccc(F)cc1]

A solution of 2-([[6-chloro-3-(4-fluorophenyl)pyridin-2-yl]methyl]amino)-4,6-bis(trifluoromethyl)pyridine-3-carbonitrile (70 mg) in HOAc (1.0 mL) was stirred overnight at 140° C. and then the mixture was concentrated under reduced pressure. The residue was purified by Prep-TLC (2:1, PE:EtOAc) to afford the title compound (22 mg) as a pink solid. ¹H NMR (400 MHz; DMSO-d₆): δ 11.58 (s, 1H), 8.33 (s, 1H), 7.43 (s, 1H), 7.39-7.32 (m, 3H), 7.23 (t, 2H), 6.36 (d, 1H), 4.53 (d, 2H) ppm. m/z 457 (M+H⁺).

### Example 24

### Synthesis of 6-([[3-cyano-4,6-bis(trifluoromethyl)pyridin-2-yl]amino]methyl)-5-(4-fluorophenyl)pyridine-2-carbonitrile

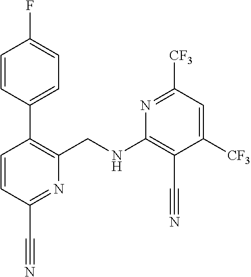 [SMILES: N#Cc1ccc(-c2ccc(F)cc2)c(CNc2nc(C(F)(F)F)cc(C(F)(F)F)c2C#N)n1]

To a solution of 2-([[6-chloro-3-(4-fluorophenyl)pyridin-2-yl]methyl]amino)-4,6-bis(trifluoromethyl)pyridine-3-carbonitrile (Example 23, Step 3, 100 mg, 0.21 mmol) in DMF (1 mL) was added Zn(CN)₂ (49 mg, 0.42 mmol), Et₃N (43 mg, 0.42 mmol), Zn (41 mg, 0.63 mmol), and Pd(dppf)Cl₂ (15 mg, 0.021 mmol). The mixture was stirred at 100° C. for 3 hours under nitrogen atmosphere. The mixture was cooled to room temperature and diluted with EtOAc. The mixture was washed with water and the organic layer was concentrated under vacuum. The residue was purified by Prep-TLC (5:1, PE:EtOAc). The residue was further purified by Prep-HPLC [column, XBridge Prep OBD C18; mobile phase 60-80% MeCN in (0.05% NH₄OH in water)] to afford the title compound (27 mg, 27% yield) as a white solid. ¹H NMR (400 MHz; DMSO-d₆): δ 8.63 (s, 1H), 8.04 (d, 1H), 7.92 (d, 1H), 7.51-7.47 (m, 2H), 7.34-7.26 (m, 3H), 4.74 (s, 2H) ppm. m/z 466 (M+H⁺).

### Example 25

### Synthesis of 2-([[3-(4-bromophenyl)pyridin-2-yl]methyl]amino)-4,6-bis(trifluoromethyl)-pyridine-3-carbonitrile

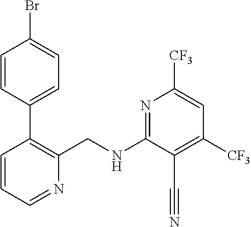 [SMILES: N#Cc1c(C(F)(F)F)cc(C(F)(F)F)nc1NCc1ncccc1-c1ccc(Br)cc1]

### Step 1: Preparation of 3-(4-bromophenyl)pyridine-2-carbonitrile

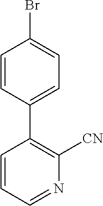 [SMILES: N#Cc1ncccc1-c1ccc(Br)cc1]

To a solution of 3-bromopyridine-2-carbonitrile (915 mg, 5.0 mmol) in 1,4-dioxane (7.2 mL) and H₂O (1.8 mL) was added 4-bromophenylboric acid (1.2 mg, 6.0 mmol), K₂CO₃ (1.4 mg, 10.0 mmol) and Pd(dppf)Cl₂ (366 mg, 0.5 mmol). The mixture was stirred for 4 h at 80° C. and then diluted with water. The mixture was extracted with EtOAc and the combined organic layers were concentrated under reduced pressure. The residue was purified by silica gel chromatography (eluent: 17% EtOAc in PE) to afford the title compound (1 g, 64% yield) as an off-white solid.

### Step 2: Preparation of 1-[3-(4-bromophenyl)pyridin-2-yl]methanamine

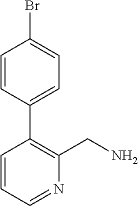 [SMILES: NCc1ncccc1-c1ccc(Br)cc1]

To a solution of 3-(4-bromophenyl)pyridine-2-carbonitrile (960 mg, 3.7 mmol) in THE (9.6 mL) was added BH₃ (2 M in THF, 7.4 mL, 14.8 mmol). The mixture was stirred for 0.5 hour at 0° C. and then at 40° C. for 2 h. The mixture was cooled to rt and MeOH was added. The mixture was concentrated under reduced pressure and the residue was purified by Prep-TLC (30:1, DCM:MeOH) to afford the title compound (340 mg, 28% yield) as a black oil.

### Step 3: Preparation of 2-([[3-(4-bromophenyl)pyridin-2-yl]methyl]amino)-4,6-bis(trifluoromethyl)pyridine-3-carbonitrile

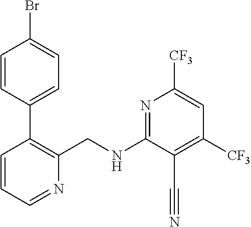 [SMILES: N#Cc1c(C(F)(F)F)cc(C(F)(F)F)nc1NCc1ncccc1-c1ccc(Br)cc1]

The title compound was prepared using General Procedure F employing 2-chloro-4,6-bis(trifluoromethyl)pyridine-3-carbonitrile (Intermediate A) and 1-[3-(4-bromophenyl)pyridin-2-yl]methanamine. The mixture was stirred for 4 h at room temperature and then diluted with water. The aqueous layer was extracted with EtOAc and the combined organic layers were concentrated under reduced pressure. The residue was purified by Prep-HPLC [column, XBridge Prep OBD C18; mobile phase 67-87% MeCN in (0.05% NH₄OH in water)] to afford the title compound as a white solid. ¹H NMR (300 MHz; DMSO-d₆): δ 8.57-8.52 (m, 2H), 7.68-7.60 (m, 3H), 7.42-7.33 (m, 4H), 4.71 (s, 2H) ppm. m/z 501 (M+H⁺).

### Example 26

### Synthesis of 2-(((1-(4-fluorophenyl)-1H-1,2,4-triazol-5-yl)methyl)amino)-4,6-bis(trifluoromethyl)nicotinonitrile

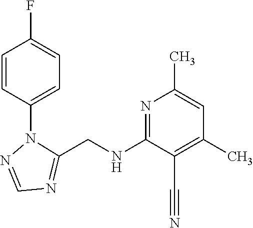 [SMILES: Cc1cc(C)c(C#N)c(NCc2ncnn2-c2ccc(F)cc2)n1]

### Step 1: Preparation of 4-fluorobenzenediazonium tetrafluoroborate

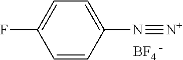

To a solution of 4-fluoroaniline (20 g, 180 mmol) in EtOH (54 mL) was added a solution of tetrafluoroboric acid (40% in water, 79 g, 360 mmol). The mixture was stirred 5 min at rt and then cooled to 0° C. Then t-BuONO (41 g, 360 mmol) was added dropwise at 0° C. and the mixture was warmed to rt and stirred for 1.5 h. The mixture was diluted with MTBE and stirred at rt for 20 min. The mixture was filtered and the solid was washed with MTBE and dried to give the title compound (29 g, 75% yield) as a white solid.

### Step 2: Preparation of ethyl 1-(4-fluorophenyl)-1H-1,2,4-triazole-5-carboxylate

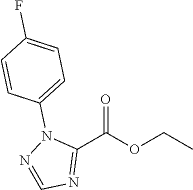 [SMILES: CCOC(=O)c1ncnn1-c1ccc(F)cc1]

To a solution of ethyl 2-isocyanoacetate (18 g, 161 mmol) in THE (280 mL) was added Cu(OAc)₂ (24 g, 134 mmol) and LiOAc (17.7 g, 269 mmol). The mixture was cooled to 0° C. and then 4-fluorobenzenediazonium tetrafluoroborate (28.2 g, 134 mmol) was added in portions at 0° C. The mixture was stirred at 0° C. for 6 h and then warmed to rt and stirred at rt for 11 h. The mixture was poured into a brine solution and extracted with EtOAc. The combined organic layers were concentrated, and the residue was purified by silica gel column chromatography (eluent: 5-17% EtOAc in PE) to give the title compound (1.4 g, 4% yield) as a yellow solid.

### Step 3: Preparation of (1-(4-fluorophenyl)-1H-1,2,4-triazol-5-yl)methanol

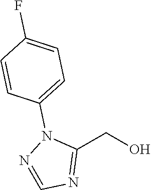 [SMILES: OCc1ncnn1-c1ccc(F)cc1]

To a solution of ethyl 2-(4-fluorophenyl)-1,2,4-triazole-3-carboxylate (1.25 mg, 5.3 mmol) in THE (25 mL) was added NaBH₄ (161 mg, 4.3 mmol) in portions at rt. The mixture was stirred at rt for 5 h and then poured into a saturated solution of NH₄Cl. The mixture was extracted with EtOAc and the combined organic layers were concentrated. The residue was triturated with DCM to give the title compound (442 mg, 43% yield) as a white solid.

### Step 4: Preparation of 5-(chloromethyl)-1-(4-fluorophenyl)-1,2,4-triazole

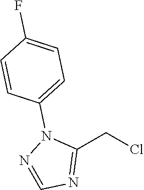 [SMILES: Fc1ccc(-n2ncnc2CCl)cc1]

The title compound was prepared using General Procedure B employing [2-(4-fluorophenyl)-1,2,4-triazol-3-yl]methanol. The mixture was diluted with MTBE and filtered to give the title as a white solid.

### Step 5: Preparation of 2-[[2-(4-fluorophenyl)-1,2,4-triazol-3-yl]methyl]isoindole-1,3-dione

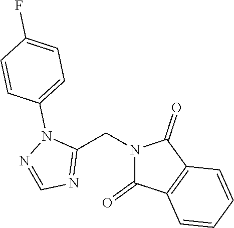 [SMILES: O=C1c2ccccc2C(=O)N1Cc1ncnn1-c1ccc(F)cc1]

The title compound was prepared using General Procedure D employing 5-(chloro-methyl)-1-(4-fluorophenyl)-1,2,4-triazole. The mixture was stirred at 50° C. for 2 h and then cooled to rt and diluted with water. The mixture was stirred at rt for 30 min and then filtered. The solid was dried to give the title compound (630 mg, 94% yield) as a white solid.

### Step 6: Preparation of 1-[2-(4-fluorophenyl)-1,2,4-triazol-3-yl]methanamine

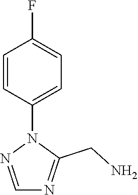 [SMILES: NCc1ncnn1-c1ccc(F)cc1]

The title compound was prepared using General Procedure E employing 2-[[2-(4-fluorophenyl)-1,2,4-triazol-3-yl]methyl]isoindole-1,3-dione. The mixture was cooled to rt and diluted with EtOH. The mixture was stirred at rt for 15 minutes and filtered. The filtrate was concentrated, and the residue was purified by Prep-TLC (10:1, DCM:MeOH) to give the title compound (288 mg, 79% yield) as a white solid.

### Step 7: Preparation of 2-(((1-(4-fluorophenyl)-1H-1,2,4-triazol-5-yl)methyl)amino)-4,6-bis(trifluoromethyl)nicotinonitrile

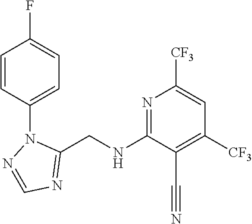 [SMILES: N#Cc1c(C(F)(F)F)cc(C(F)(F)F)nc1NCc1ncnn1-c1ccc(F)cc1]

The title compound was prepared using General Procedure F employing 2-chloro-4,6-bis(trifluoromethyl)pyridine-3-carbonitrile (Intermediate A) and 1-[2-(4-fluorophenyl)-1,2,4-triazol-3-yl]. The mixture was cooled to rt and diluted with EtOAc and washed with 1M LiCl. The organic layer was concentrated under reduced pressure and the residue was purified by prep-TLC (20:1, DCM:MeOH) to give the title compound (80 mg, 71% yield) as a white solid. ¹H NMR (300 MHz; DMSO-d₆): δ 8.86 (t, 1H), 8.05 (s, 1H), 7.63-7.59 (m, 2H), 7.42-7.36 (m, 3H), 4.79 (d, 2H). m/z 431 (M+H⁺).

### Example 27

### Synthesis of 2-([[3-(4-fluorophenyl)-6-methoxypyridin-2-yl]methyl]amino)-4,6-bis(trifluoromethyl)pyridine-3-carbonitrile

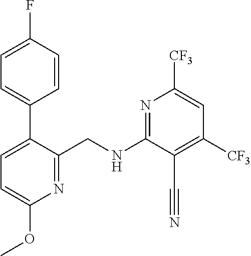 [SMILES: COc1ccc(-c2ccc(F)cc2)c(CNc2nc(C(F)(F)F)cc(C(F)(F)F)c2C#N)n1]

### Step 1: Preparation of 3-(4-fluorophenyl)-6-methoxypyridine-2-carbonitrile

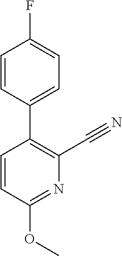 [SMILES: COc1ccc(-c2ccc(F)cc2)c(C#N)n1]

To a stirred solution of 6-chloro-3-(4-fluorophenyl)pyridine-2-carbonitrile (Example 23, Step 1, 2.9 g, 12.3 mmol) in DMF (29 mL) was added MeONa (2.7 g, 49.2 mmol) at rt. The mixture was stirred for 4 h at 50° C. and after cooling to to rt and a saturated solution of NH₄Cl (aq.) was added. The mixture was extracted with EtOAc and the combined organic layers were washed with water and concentrated under reduced pressure. The residue was purified by silica gel column chromatography (eluent: 16% EtOAc in PE) to afford the title compound (1.3 g, 39% yield) as a white solid.

### Step 2: Preparation of 1-[3-(4-fluorophenyl)-6-methoxypyridin-2-yl]methanamine

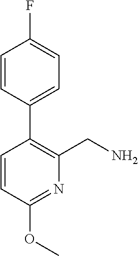 [SMILES: COc1ccc(-c2ccc(F)cc2)c(CN)n1]

To a stirred solution of 3-(4-fluorophenyl)-6-methoxypyridine-2-carbonitrile (500 mg, 2.2 mmol) in MeOH (50 mL) was added 10% Pd/C (14 mg, 0.131 mmol) at room temperature. The mixture was stirred at rt for 1 h under hydrogen atmosphere. The mixture was filtered and the filtrate was concentrated under vacuum to afford the title compound as a light-yellow crude oil, which was used in the next step without further purification.

### Step 3: Preparation of 2-([[3-(4-fluorophenyl)-6-methoxypyridin-2-yl]methyl]amino)-4,6-bis(trifluoromethyl)pyridine-3-carbonitrile

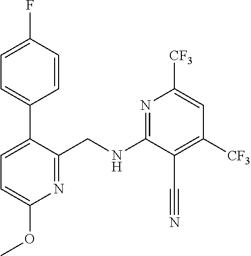 [SMILES: COc1ccc(-c2ccc(F)cc2)c(CNc2nc(C(F)(F)F)cc(C(F)(F)F)c2C#N)n1]

The title compound was prepared using General Procedure F employing 2-chloro-4,6-bis(trifluoromethyl)pyridine-3-carbonitrile (Intermediate A) and 1-[3-(4-fluorophenyl)-6-methoxypyridin-2-yl]methanamine. The mixture was stirred for 1 h at rt and then diluted with EtOAc and washed with water. The organic layer was concentrated under vacuum and the residue was purified by Prep-TLC (5:1, PE:EtOAc) to afford the title compound (73 mg, 32% yield) as a white solid. ¹H NMR (300 MHz; DMSO-d₆): δ 8.37 (t, 1H), 7.61 (d, 1H), 7.48-7.39 (m, 3H), 7.32-7.24 (m, 2H), 6.80 (d, 1H), 4.65 (d, 2H), 3.77 (s, 3H) ppm. m/z 471 (M+H⁺).

### BIOLOGICAL EXAMPLES

### Example 1

The ability of the compounds of Formula (I) to inhibit polymerase activity of Pol theta was determined using the assay described below.

A mixture of 20 uL of Pol theta polymerase domain (residues 1819-2590) at a final concentration of 4 nM in assay buffer (20 m M TRIS, pH 7.80, 50 mM KCl, 10 mM MgCl₂, 1 mM DTT, 0.01% BSA, 0.01% Tween20) was added to test compounds (11-point dilution series of test compounds) except the low control wells without test compounds. The above enzyme and test compound inhibitor mixture was then incubated at room temperature for 15 min. An equal volume (20 μl) of dNTP substrate mix (48 μM) and primed molecular beacon DNA (obtained by annealing template SEQ ID NO 2: (5′-CCTTCCTCCCGTGTCTTG-TACCTTCCCGTCA-GGAGGAAGG-3′) with 5′-TAMRA and 3′-BHQ and primer DNA (SEQ ID NO 3: 5′-GACGGGAAGG-3′) in 10 mM Tris-HCl pH 8.0, 100 mM NaCl buffer) (96 nM) in assay buffer was added to all the test wells. The inhibition activity was measured by monitoring the fluorescence change over 30 min at 535 nm upon excitation at 485 nm. The high control (DMSO with enzyme) with high fluorescence intensity represents no inhibition of polymerase reaction while the low control (DMSO with buffer) with low fluorescence intensity represents full inhibition of polymerase activity. Slope of the reaction progress curves were used to calculate the rate of polymerization. The rates were used to determine the percent inhibition using a four-parameter inhibition model to generate IC₅₀, Hill slope and max inhibition.

The IC₅₀ of the compounds in Table 1 above are disclosed in Table 2 below: (+) IC₅₀=10 uM-1 uM; (++) IC₅₀=1 uM-500 nM; (+++) IC₅₀=500 nM-200 nM; (++++) IC₅₀<200 nM

| | Primer |
|---|---|
| | extension |
| Cpd. | Assay IC₅₀ |
| # | (uM) |
| 1 | ++ |
| 2 | ++++ |
| 3 | ++++ |
| 4 | ++++ |
| 5 | ++++ |
| 6 | ++++ |
| 7 | ++++ |
| 8 | ++++ |
| 9 | ++++ |
| 10 | ++++ |
| 11 | ++++ |
| 12 | ++++ |
| 13 | ++++ |
| 14 | + |
| 15 | + |
| 16 | ++++ |
| 17 | ++ |
| 18 | + |
| 19 | ++++ |
| 20 | ++++ |
| 21 | +++ |
| 22 | ++++ |
| 23 | ++++ |
| 24 | ++++ |
| 25 | ++++ |
| 26 | +++ |
| 27 | + |

What is claimed:

1. A compound of Formula (I): 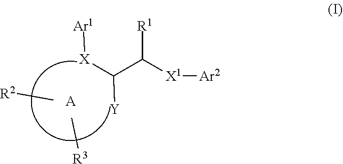
wherein:
X¹ is —NH— or —O—;
A is:
(i) a five membered heteroaryl ring wherein X is C or N, Y is O or N, and the heteroaryl may contain an additional nitrogen atom; or
(ii) a six-membered heteroaryl ring wherein X is C, Y is N, and the heteroaryl may contain one or two additional nitrogen atoms;
Ar¹ is phenyl, heteroaryl, or heterocyclyl, wherein each aforementioned ring is substituted with R^{a}, R^{b}, and/or R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂;
Ar² is phenyl or heteroaryl, wherein said phenyl and heteroaryl are substituted with R^{d} and further substituted with R^{e} and/or R^{f}, wherein R^{d} is haloalkyl, and R^{e} and R^{f} are independently selected from hydrogen, alkyl, halo, haloalkyl, alkoxy, haloalkoxy, hydroxy, cyano, cyanomethyl, aminocarbonylmethyl, heteroaryl, or heterocyclyl, wherein said heteroaryl and heterocyclyl of R^{e} and/or R^{f} are unsubstituted or substituted with one, two, or three substituents independently selected from alkyl, halo, haloalkyl, and hydroxy;
R¹ is hydrogen, alkyl, hydroxyalkyl, alkoxyalkyl, aminoalkyl, aminocarbonylalkyl, or phenalkyl, wherein phenyl in phenalkyl is substituted with R^{g}, R^{h}, and Rⁱ, wherein R^{g}, R^{h}, and Rⁱ are independently selected from hydrogen, alkyl, halo, haloalkyl, alkoxy, haloalkoxy, hydroxy, and cyano; and
R² and R³ are independently hydrogen, alkyl, halo, haloalkyl, haloalkoxy, cyano, or —CONH₂; or
a pharmaceutically acceptable salt thereof; provided that the compound of Formula (I) is not:
2-pyridinamine, N-[(4-phenyl-4H-1,2,4-triazol-3-yl)methyl]-6-(trifluoromethyl)-;
5-(((5-bromo-6-(trifluoromethyl)pyridin-2-yl)oxy)methyl)-3-(chloromethyl)-4-(6-chloropyridin-3-yl)isoxazole;
2-pyrimidinamine, N-[(4-phenyl-4H-1,2,4-triazol-3-yl)methyl]-4-(trifluoromethyl)-;
2-pyridinamine, N-[(4-phenyl-4H-1,2,4-triazol-3-yl)methyl]-5-(trifluoromethyl)-;
pyrimidine, 4-[[[3-fluoro-5-(trifluoromethyl)-2-pyridinyl]oxy]methyl]-5-phenyl-;
1,4′-bi-1H-pyrazole, 4-iodo-1′-methyl-5′-[[4-(trifluoromethyl)phenoxy]methyl]-;
3H-1,2,4-triazol-3-one, 2,4-dihydro-5-methoxy-2-methyl-4-[2-[1-[[4-(trifluoromethyl)-2-pyridinyl]oxy]ethyl]-3-pyridinyl]-;
3(2H)-isoxazolone, 5-methoxy-2-methyl-4-[2-[1-[3-(trifluoromethyl)phenoxy]ethyl]-3-pyridinyl]-;
3H-1,2,4-triazol-3-one, 2,4-dihydro-5-methoxy-2-methyl-4-[2-[[[4-(trifluoromethyl)-2-pyridinyl]oxy]methyl]-3-pyridinyl]-;
3(2H)-isoxazolone, 5-methoxy-2-methyl-4-[2-[[3-(trifluoromethyl)phenoxy]methyl]-3-pyridinyl]-;
3(2H)-isoxazolone, 5-methoxy-2-methyl-4-[2-[1-[[4-(trifluoromethyl)-2-pyridinyl]oxy]ethyl]-3-pyridinyl]-;
3(2H)-isoxazolone, 5-methoxy-2-methyl-4-[2-[[[4-(trifluoromethyl)-2-pyridinyl]oxy]methyl]-3-pyridinyl]-;
3H-1,2,4-triazol-3-one, 2,4-dihydro-5-methoxy-2-methyl-4-[2-[1-[3-(trifluoromethyl)phenoxy]ethyl]-3-pyridinyl]-;
3H-1,2,4-triazol-3-one, 2,4-dihydro-5-methoxy-2-methyl-4-[2-[[3-(trifluoromethyl)phenoxy]methyl]-3-pyridinyl]-; or
4-pyrimidinamine, N-[(5-phenyl-4-oxazolyl)methyl]-2-(2-pyridinyl)-6-(trifluoromethyl)-; or a pharmaceutically acceptable salt thereof.

2. (canceled)

3. The compound of claim 1, wherein Ar² is a six-membered heteroaryl substituted with R^{d} and R^{e} and R^{f}, where R^{d} is haloalkyl.

4. The compound of claim 1, wherein Ar² is pyridinyl substituted with R^{d} and R^{e} and R^{f}, where R^{d} is haloalkyl.

5. The compound of claim 1, wherein Ar² is pyridin-2-yl substituted with R^{d} and R^{e} and/or R^{f}, where R^{d} is difluoromethyl or trifluoromethyl, R^{e} is haloalkyl, alkoxy, halo, haloalkoxy, hydroxy, or cyano, and R is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R are unsubstituted or substituted with one, two, or three substituents independently selected from alkyl, halo, haloalkyl, and hydroxy.

6. The compound of claim 1, wherein Ar² is phenyl substituted with R^{d} and R^{e} and R^{f}, where R^{d} is haloalkyl.

7. The compound of claim 1, wherein Ar² is phenyl substituted with R^{d} and R^{e} and/or R^{f}, where R^{d} is difluoromethyl or trifluoromethyl, R^{e} is haloalkyl, alkoxy, halo, haloalkoxy, hydroxy, or cyano, and R is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R are unsubstituted or substituted with one, two, or three substituents independently selected from alkyl, halo, haloalkyl, and hydroxy.

8. The compound of claim 1, wherein R¹ is hydrogen, methyl, hydroxymethyl, 2-hydroxyethyl, 4-hydroxybenzyl, or aminocarbonylmethyl.

9. The compound of claim 1, wherein R¹ is hydrogen.

10. The compound of claim 1, wherein ring A is a five-membered heteroaryl ring.

11. The compound of claim 1, wherein ring A is a ring of formula (i) to (v):

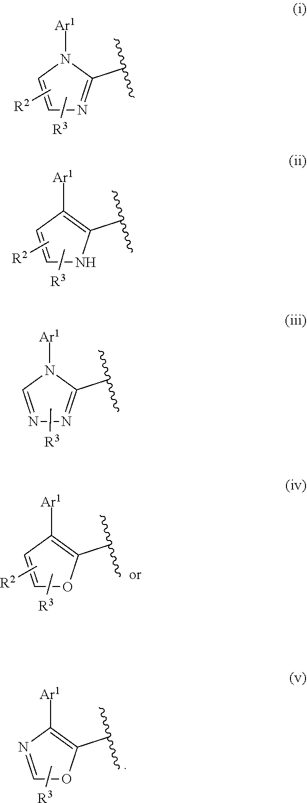

12. The compound of claim 11, wherein ring A has formula (i) or (iii).

13. The compound of claim 1, wherein ring A is a six-membered heteroaryl ring.

14. The compound of claim 13, wherein ring A is a ring of formula (ia) to (ic):

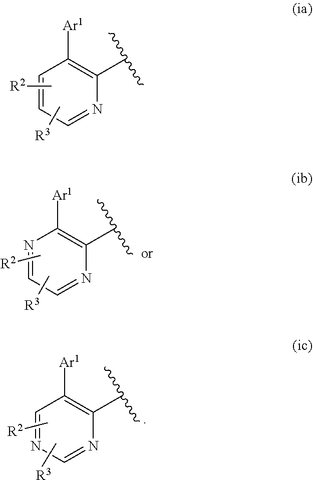

15. (canceled)

16. The compound of claim 1, wherein Ar¹ is phenyl, wherein said phenyl is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂.

17. The compound of claim 16, wherein Ar¹ is substituted with R^{a}, R^{b}, and R^{c}, wherein R^{a}, R^{b}, and R^{c} are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy.

18. The compound of claim 1, wherein Ar¹ is heteroaryl, wherein said heteroaryl is substituted with R^{a}, R^{b}, and R^{c}, wherein R^{a}, R^{b}, and R^{c} are independently selected from hydrogen, alkyl, cycloalkyl, cycloalkoxy, halo, haloalkyl, alkoxy, haloalkoxy, hydroxy, cyano, and —CONH₂.

19. The compound of claim 1, wherein X¹ is NH.

20. The compound of claim 1, wherein X₁ is O.

21. The compound of claim 1, wherein R² and R³ are independently selected from hydrogen, methyl, fluoro, chloro, trifluoromethyl, trifluoromethoxy, or cyano.

22. The compound of claim 1, wherein R² is hydrogen and R³ is selected from hydrogen, methyl, fluoro, chloro, trifluoromethyl, trifluoromethoxy, or cyano.


SEQUENCE LISTING (3 sequences)

SEQ ID NO: 1 - DNA, 40 bp, Artificial sequence
  Synthetic polynucleotide
ccttcctcccgtgtcttgtaccttcccgtcaggaggaagg

SEQ ID NO: 2 - DNA, 40 bp, Artificial sequence; identical to SEQ ID NO: 1
  Synthetic polynucleotide
  misc_feature (1)..(1): 5′ TAMRA
  misc_feature (40)..(40): 3′ BHQ

SEQ ID NO: 3 - DNA, 10 bp, Artificial sequence
  Synthetic polynucleotide
gacgggaagg